SEQUENCE LISTING (27 sequences)

SEQ ID NO: 1 - DNA, 34 bp, Artificial Sequence
  Synthetic
attatccatggtgaatgaatgaatggggaaataa

SEQ ID NO: 2 - DNA, 33 bp, Artificial Sequence
  Synthetic
tatttatttccccattcattcattcaccatgga

SEQ ID NO: 3 - DNA, 26 bp, Artificial Sequence
  Synthetic
atcggggggggggggggggggggcta

SEQ ID NO: 4 - DNA, 26 bp, Artificial Sequence
  Synthetic
tagccccccccccccccccccccgat

SEQ ID NO: 5 - DNA, 8 bp, Artificial Sequence
  Synthetic
atgggaag

SEQ ID NO: 6 - DNA, 11 bp, Artificial Sequence
  Synthetic
gccacccctca

SEQ ID NO: 7 - DNA, 13 bp, Artificial Sequence
  Synthetic
atgacccccctca

SEQ ID NO: 8 - DNA, 16 bp, Artificial Sequence
  Synthetic
cgttccccttaaataa

SEQ ID NO: 9 - DNA, 14 bp, Artificial Sequence
  Synthetic
gtctggggggtatg

SEQ ID NO: 10 - DNA, 16 bp, Artificial Sequence
  Synthetic
ataacaaaaaatttcc

SEQ ID NO: 11 - DNA, 17 bp, Artificial Sequence
  Synthetic
agtcaccccccaactaa

SEQ ID NO: 12 - DNA, 16 bp, Artificial Sequence
  Synthetic
ttattttcccctccca

SEQ ID NO: 13 - DNA, 23 bp, Artificial Sequence
  Synthetic
aaccccaaagacaccccccacag

SEQ ID NO: 14 - DNA, 12 bp, Artificial Sequence
  Synthetic
acgatcaaaagg

SEQ ID NO: 15 - DNA, 10 bp, Artificial Sequence
  Synthetic
catagcacat

SEQ ID NO: 16 - DNA, 9 bp, Artificial Sequence
  Synthetic
agggccata

SEQ ID NO: 17 - DNA, 12 bp, Artificial Sequence
  Synthetic
ttgcggacgctg

SEQ ID NO: 18 - DNA, 14 bp, Artificial Sequence
  Synthetic
gaacatacctacta

SEQ ID NO: 19 - DNA, 12 bp, Artificial Sequence
  Synthetic
ttaattgattaa

SEQ ID NO: 20 - DNA, 882 bp, Staphylococcus aureus
gcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaattaa

SEQ ID NO: 21 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 22 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGDIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 23 - DNA, 13 bp, Artificial Sequence
  Synthetic
acagccgctttcc

SEQ ID NO: 24 - DNA, 22 bp, Artificial Sequence
  Synthetic
aaccccccccctccccccgctt

SEQ ID NO: 25 - DNA, 11 bp, Artificial Sequence
  Synthetic
caaagggacaa

SEQ ID NO: 26 - DNA, 12 bp, Artificial Sequence
  Synthetic
caaaaggaacaa

SEQ ID NO: 27 - DNA, 20 bp, Artificial Sequence
  Synthetic
atcgatcgatcgatcgatcg

(19) United States
(12) Patent Application Publication
(10) Pub. No.: US 2018/0164280 A1
(43) Pub. Date: Jun. 14, 2018

(54) MODIFIED NUCLEIC ACIDS FOR NANOPORE ANALYSIS

(71) Applicant: Ibis Biosciences, Inc., Carlsbad, CA (US)

(72) Inventors: David J. Ecker, Carlsbad, CA (US); Stanley T. Motley, Carlsbad, CA (US); James C. Hannis, Carlsbad, CA (US); Lara G. Krieg, Carlsbad, CA (US); Todd P. Michael, Carlsbad, CA (US); David D. Duncan, Carlsbad, CA (US); Shane G. Poplawski, Carlsbad, CA (US); Thomas N. Chiesl, Carlsbad, CA (US)

(21) Appl. No.: 15/804,711

(22) Filed: Nov. 6, 2017

Related U.S. Application Data
(60) Provisional application No. 62/418,490, filed on Nov. 7, 2016.

(51) Int. Cl. G01N 33/487; C12Q 1/6806; C12Q 1/6869

(52) U.S. Cl. CPC G01N 33/48721; C12Q 1/6806; C12Q 1/6869; C12Q 2525/113; B82Y 5/00; C12Q 2525/119; C12Q 2525/117; C12Q 2535/10; C12Q 2525/191

(57) ABSTRACT

Provided herein is technology relating to nanopore analysis of nucleic acids and particularly, but not exclusively, to compositions, methods, systems, and kits for analysis of nucleic acids or other assays comprising nucleic acid components.

This application claims priority to U.S. Provisional Patent Application No. 62/418,490, filed Nov. 7, 2016, which is incorporated herein by reference in its entirety.

### FIELD

Provided herein is technology relating to nanopore analysis of nucleic acids and particularly, but not exclusively, to compositions, methods, systems, and kits for analysis of nucleic acids or other assays comprising nucleic acid components.

### BACKGROUND

Nanopore sequencing technologies rely on small electrical (e.g., current, resistance, conductance, voltage) variations associated with one or more nucleotides translocating through a nanopore. Often, the nucleic acid does not move smoothly through the nanopore, but is subjected to slipping, delay, and other aberrant motions through the nanopore that produce electrical variations and errors in the measurements. Consequently, the complex and interpretative modeling of the electrical variation yields nanopore sequencing error rates that are exceedingly high. The high sequencing error rates prohibit the accurate determination of several types of nucleic acid sequences. As an example, current commercial nanopore sequencers cannot accurately determine the number of nucleotide repeats within a repeating sequence. This ambiguity has major consequences to the medical and forensic fields where repeat length is indicative of a medical disease state or is used for identity determination in forensics. Alternative approaches to nanopore analysis of nucleic acids are needed.

### SUMMARY

In many instances where sequencing fails, analysis of nucleic acids based on base counting or base composition succeeds. For example, as described herein, use of modified nucleotides in nucleic acids provides a nanopore technology for analysis of nucleic acids that provides information about a nucleic acid (e.g., base composition, base number, presence or absence of a single nucleotide polymorphism (SNP), number of short repeat sequences, etc.) without determining full nucleotide sequence information. Additionally, the electrical signatures for modified nucleotides are significantly distinct, which minimizes and/or eliminates the complex modeling associated with direct nanopore sequencing.

In some embodiments, the technology provided herein improves and/or modifies nanopore sensing technology, such as technologies based on solid-state nanopores or protein-based pores (e.g., such as those made by Oxford Nanopore Technologies). In particular, the technology described herein is based on using modified nucleotides that are used to distinguish nucleic acid (e.g., DNA or RNA) sequences in a sample. During the development of embodiments of the technology described herein, experimental data were collected indicating that a modified nucleotide in a nucleic acid provides a significant perturbation in the monitored nanopore current and/or conductance relative to unmodified nucleotides. In addition, experiments were conducted in which nucleic acids were analyzed using the technology to determine base counts, base composition, and repeat length. In further experiments conducted during the development of embodiments of the technology, unique sequence tags were created and read during passage through the nanopore. The data produced in these experiments required less rigorous processing to characterize a nucleic acid (e.g., by base count, base composition, repeat length, etc.) than in nanopore sequencing experiments that attempt to provide accurate nucleotide sequence information. Additionally, the deviation in the nanopore current and/or conductance (e.g., duration and differential magnitude) can be controlled by the size, structure, and charge of the nucleic acid modification provided in the nucleic acid under analysis. The technology finds use, e.g., in nucleic acid diagnostic and forensic assays that are based on base count, base composition, repeat length, etc. and that do not necessarily depend on nucleotide sequence determination, though some embodiments provide information about nucleic acids to supplement nucleotide sequence determination.

Accordingly, provided herein is technology related to a method for characterizing a nucleic acid, the method comprising steps of modifying the nucleic acid to provide a modified nucleic acid; translocating the modified nucleic acid through a nanopore; measuring an electrical signal produced by translocation of the modified nucleic acid through the nanopore; and characterizing the nucleic acid by analyzing the electrical signal.

In some embodiments, characterizing the nucleic acid does not comprise determining a nucleotide sequence of the nucleic acid. In some embodiments, characterizing the nucleic acid does not comprise determining the order of any of the bases and/or nucleotides in the nucleic acid. In some embodiments, characterizing the nucleic acid does not comprise determining the position of any base or nucleotide relative to any other base or nucleotide. In some embodiments, characterizing the nucleic acid does not comprise determining the absolute position of any base or nucleotide in the nucleic acid. In some embodiments, characterizing the nucleic acid does not comprise determining the position of any base or nucleotide relative to the 3′ and/or 5′ end of the nucleic acid. In some embodiments, characterizing the nucleic acid does not comprise determining the order, relative position, or absolute position of any of the four bases A, C, G, T, or U in a nucleic acid. In some embodiments, characterizing the nucleic acid does not comprise determining the order, relative position, or absolute position of pyrimidines and/or purines in the nucleic acid. In some embodiments, characterizing the nucleic acid does not comprise determining the order, relative position, or absolute position of nucleotides or bases that form three-hydrogen bond base pairs (e.g., C and G) with respect to the order, relative position, or absolute position of nucleotides or bases that form two-hydrogen bond base pairs (e.g., A, T, and U). In some embodiments, characterizing the nucleic acid does not comprise determining the order, relative position, or absolute position of any modification (e.g., label (e.g., fluorescent label, radioactive label, biotin label, antibody label, spin label, isotopic label, chemical label, functional group label, mass label, magnetic label, quantum dot label, etc.), methylation, chemical modification, etc.) of any nucleotide or base in the nucleic acid. In some embodiments, characterizing the nucleic acid does not comprise determining the order, relative position, or absolute position of any modification (e.g., phosphorothioate bond, peptide bond), damage, break, nick, etc. of the nucleic acid backbone. In some embodiments, characterizing the nucleic acid does not comprise determining the order, relative position, or absolute position of any nuclease recognition site (e.g., restriction enzyme recognition site). In some embodiments, characterizing the nucleic acid does not comprise determining the size (e.g., number of nucleotides or mass) of the nucleic acid or any fragment produced from the nucleic acid.

In particular embodiments, the electrical signal is measured as a function of time. In particular embodiments, the electrical signal indicates the presence of the modified nucleic acid in the nanopore. The technology is not limited with respect to the electrical signal that is measured. For instance, in some embodiments the electrical signal is current; in some embodiments, the electrical signal is impedance, conductance, or resistance. In some embodiments, the method further comprises a step of providing a voltage across the nanopore.

The methods relate to use of modified nucleic acids and/or modified nucleotides. For instance, in some embodiments, modifying the nucleic acid comprises modifying a nucleotide of the nucleic acid (e.g., linking a chemical moiety to the nucleotide; linking a dye to the nucleotide; and/or removing a base to produce an abasic site) or incorporating a modified nucleotide into the nucleic acid. In some embodiments, modifying the nucleic acid comprises modifying a linkage between nucleotides of the nucleic acid. In some embodiments comprising modifying the nucleic acid, modifying the nucleic acid comprises providing a linker between nucleotides of the nucleic acid; providing an uncharged linkage between nucleotides of the nucleic acid; and/or providing peptide nucleic acid linkage between nucleotides of the nucleic acid.

In some embodiments, producing a modified nucleic acid comprises use of amplification primers (e.g., for polymerase chain reaction, linear chain reaction, reverse transcription polymerase chain reaction, real-time polymerase chain reaction, etc.) that are modified according to the technology provided herein, e.g., one or more primers comprises a modification that is detectable by a nanopore. Embodiments provide technologies comprising use of 1, 2, 3, 4, 5, 6, 7, 8, 9, 10, 11, 12, 13, 14, 15, 16, 17, 18, 19, 20 or more primers, each of which comprises a modification that, in some embodiments, is the same as one or more modifications of one or more other primers and/or that, in some embodiments, is different from one or more modifications of one or more other primers.

In some embodiments, modified nucleic acids are produced by an amplification reaction (e.g., polymerase chain reaction, linear chain reaction, reverse transcription polymerase chain reaction, real-time polymerase chain reaction, etc.). In some embodiments, a modified nucleotide is introduced into a nucleic acid by an amplification reaction (e.g., an amplification reaction is performed with one or more modified nucleotide(s) that is/are incorporated into the amplicon during the synthesis step(s) of the amplification reaction). In some embodiments, a precursor of a modified nucleotide is introduced into a nucleic acid by an amplification reaction (e.g., an amplification reaction is performed with one or more precursors of a modified nucleotide that is/are incorporated into the amplicon during the extension (e.g., synthesis) step(s) of the amplification reaction). Then, in some embodiments, the modified nucleotide is produced in the nucleic acid by a chemical reaction that converts the precursor of a modified nucleotide to a modified nucleotide.

In some embodiments of the technology, a change in the magnitude of the electrical signal or a change in the electrical signal in the time domain indicates the presence of the modified nucleotide in the nanopore. And, in some embodiments, characterizing the nucleic acid comprises identifying the presence of repeats in the nucleic acid. In some embodiments, characterizing the nucleic acid comprises counting the number of repeats in the nucleic acid; identifying the presence of a single nucleotide polymorphism in the nucleic acid; and/or identifying the presence of a modified base in the nucleic acid.

Some embodiments of the technology provide compositions, e.g., reaction mixtures. For example, some embodiments provide a reaction mixture comprising a modified nucleic acid and a nanopore. In some embodiments of compositions (e.g., reaction mixtures), the modified nucleic acid comprises a modified nucleotide. In some embodiments of compositions (e.g., reaction mixtures), the modified nucleic acid comprises a modified linkage between two nucleotides (e.g., the nucleic acid backbone is modified). In some embodiments of compositions (e.g., reaction mixtures), the modified nucleic acid comprises a chemical linker between two nucleotides. In some embodiments of compositions (e.g., reaction mixtures), the modified nucleic acid comprises an abasic site. In some embodiments of compositions (e.g., reaction mixtures), the modified nucleic acid comprises a nucleotide modified with a covalently attached dye or other chemical moiety. In some embodiments of compositions (e.g., reaction mixtures), the nanopore comprises a protein. In some embodiments of compositions (e.g., reaction mixtures), the nanopore is a solid state nanopore. In some embodiments of compositions (e.g., reaction mixtures), a lipid bilayer comprises the nanopore.

Some embodiments of the technology provide kits for analyzing a nucleic acid. For example, in some embodiments the technology provides a kit comprising a nanopore apparatus and a composition to modify a nucleic acid. In some embodiments, the composition produces an abasic site in a nucleic acid. In some embodiments, the composition comprises uracil-DNA glycosylase or uracil N-glycosylase. In some embodiments, the composition comprises a reactive dye. In some embodiments, the composition comprises a chemical linker or spacer. In some embodiments, the composition produces a chemically-modified nucleotide. In some embodiments, the composition comprises a drag tag. In some embodiments, the nanopore comprises a protein. In some embodiments, the nanopore is a solid state nanopore. In some embodiments, a lipid bilayer comprises the nanopore. Some embodiments of kits further comprise an exonuclease, e.g., a lambda exonuclease. Some embodiments of kits provide reagents for modifying a nucleotide or a nucleic acid and some embodiments of kits provide a modified nucleotide. Accordingly, some embodiments relate to a kit comprising a nanopore and a modified nucleotide. In some embodiments, the modified nucleotide comprises a covalently attached dye.

In additional embodiments, the technology relates to a system comprising a nanopore apparatus and a composition to modify a nucleic acid. In some embodiments, the system further comprises an electrical source providing a voltage or current. In some embodiments, the system further comprises a processor configured to analyze electrical signals recorded as a function of time. In some embodiments, the system further comprises a processor configured to perform a method for characterizing a nucleic acid, e.g., as described herein. In some embodiments, the system further comprises a lipid bilayer. In some embodiments of the system, the composition of the system produces an abasic site in a nucleic acid, e.g., the composition comprises uracil-DNA glycosylase or uracil N-glycosylase. In some embodiments, the composition of the system comprises a reactive dye. In some embodiments, the composition of the system comprises a chemical linker or spacer. In some embodiments, the composition of the system produces a chemically-modified nucleotide. In some embodiments, the composition of the system comprises a drag tag. In some embodiments, the nanopore of the system comprises a protein, e.g., a solid state nanopore or a protein nanopore. In some embodiments, a lipid bilayer comprises the nanopore of the system. Some embodiments of systems further comprise an exonuclease, e.g., a lambda exonuclease. Some embodiments of systems comprise a nanopore and a modified nucleotide, e.g., a modified nucleotide comprising a covalently attached dye.

The methods, compositions (e.g., reaction mixtures), kits, and systems find use in various applications. For example, embodiments of the technology relate to use of a method, reaction mixture, kit, or system described herein to analyze a nucleic acid. Some embodiments relate to use of a method, reaction mixture, kit, or system described herein to determine the base count of a nucleic acid. Some embodiments relate to use of a method, reaction mixture, kit, or system described herein to determine the base composition of a nucleic acid. Some embodiments relate to use of a method, reaction mixture, kit, or system described herein to determine an unordered base count or unordered base composition of a nucleic acid. Some embodiments relate to use of a method, reaction mixture, kit, or system described herein s to identify a nucleic acid. Some embodiments relate to use of a method, reaction mixture, kit, or system described herein to identify a nucleic acid by a barcode. Some embodiments relate to use of a method, reaction mixture, kit, or system described herein to detect a nucleic acid.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 shows an example of a modified oligonucleotide, e.g., tested in Example 1. FIG. 1 shows a depiction of the double-stranded product comprising fluorescein-modified thymidine nucleotides in each of the GAAT tetranucleotide repeats and having four-nucleotide single-stranded overhangs with 5′ phosphates. The insert at the upper right provides the chemical structure of the fluorescein-modified thymidine nucleotide.

FIG. 2 is an illustration showing the ligation of double-stranded, repeat-modified nucleic acids to yield a long concatemer product as described in Example 1. In this exemplary illustration, eight double-stranded segments are ligated to form a concatemer. The ligated products were subsequently processed using a commercial nanopore sequencing preparation kit to join a 5′ adapter to one end and a hairpin adapter to the other end to provide a nucleic acid that is compatible with the nanopore detector.

FIG. 3 shows data collected from a nanopore analysis of the concatemer product depicted in FIG. 2. Current at the nanopore was recorded as a function of time. As shown by the signal, a significant reduction in current was observed as each fluorescein-modified thymidine passed though the nanopore. The number of fluorescein-modified thymidines was readily determined from the data, from which the number of repeats in each segment of the nucleic acid was determined. The high current peak at the beginning of the trace is produced by the commercial 5′ adapter (e.g., to initiate translocation of the nucleic acid through the nanopore) and the high current double peak is associated with the hairpin adapter. Eight ligated segments of nucleic acid are observed in the data. To the right of the hairpin adapter are the data collected for the unmodified complementary strand as it passed through the nanopore with a higher level of current in comparison to the modified nucleotides.

FIG. 4A shows that abasic sites produce a large positive peak of current when a nucleic acid comprising an abasic site translocates through a nanopore. A DNA molecule comprising an abasic site followed closely by fluorescein was produced. Introduction of abasic sites produced a signature comprising a large positive peak (1) produced by the abasic site followed by a negative peak (2) produced by the fluorescein.

FIG. 4B shows use of abasic sites in nucleic acids. Data were collected from nucleotides comprising multiple nucleotide modifications on a single molecule. Nucleic acids comprising 0 to 16 abasic sites (a “site” consisted of 3 consecutive abasic nucleotide residues) were analyzed using a nanopore device. The data indicated an increase in current as the abasic site passed through the nanopore; multiple sets of abasic sites were easily be distinguished on the same molecule.

FIG. 5 shows data collected from nucleic acids comprising consecutive modified nucleotide sites. Products containing 1 (bottom traces), 2 (middle traces), or 3 (top traces) consecutive abasic sites (5A) or fluorescein molecules (5B) were analyzed through a nanopore. The data indicated that signal magnitude and width increased with increased consecutive modifications.

FIG. 6 shows that the spacing of modifications affects the nanopore current signatures. Nucleic acids containing two consecutive abasic sites (XX, bottom traces) or two abasic sites separated by one nonmodified base (X-X, top traces) were analyzed through a nanopore. For both constructs, a double peak signal was detected (left). Analysis of the peaks indicated that the shapes of the two-peak signals for the two constructs were different (right).

FIG. 7 shows data collected from nucleic acids comprising combinatorial nucleotide modifications. Nucleic acids comprising nucleotides modified with fluorescein (F), abasic nucleotide residues (X), or combinations of nucleotides modified with fluorescein and abasic nucleotide residues produced unique signatures in nanopore currents. Nucleic acid constructs comprised each nucleotide modification pattern as a concatemer of four repeats. Nucleic acids comprising a pattern in which a nucleotide modified with fluorescein preceded an abasic site produced a more detectable signal than a pattern in which an abasic residue preceded a fluorescein-modified nucleotide.

FIG. 8 shows that TAMRA dye produces a similar signal to fluorescein in nanopore analysis of nucleic acids. A segment of DNA containing both TAMRA and fluorescein molecules was created. Alternating fluorophores in the sequence produces similar negative current peaks caused by TAMRA dye (1) and fluorescein (2).

FIG. 9 shows that a C9 spacer provides a large positive peak of current when a nucleic acid comprising a C9 spacer translocates through a nanopore. A DNA molecule comprising 3 consecutive C3 spacers (a C9 spacer) followed closely by a fluorescein-modified nucleotide was produced. Alternating C9 spacers with fluorescein-modified nucleotides produces a signature comprising a large positive peak (1) produced by the spacer followed by a negative peak (2) produced by the fluorescein.

FIG. 10 shows that a PEG18 spacer produces a large positive peak of current when a nucleic acid comprising a PEG18 spacer translocates through a nanopore. A DNA molecule comprising a PEG18 spacer followed closely by a fluorescein-modified nucleotide was produced. Alternating PEG18 spacers with fluorescein-modified nucleotides produces signature comprising a large positive peak (1) produced by the spacer followed by a negative peak (2) produced by fluorescein.

FIG. 11 shows that chemical modification of nucleotides with azide produces a positive signal relative to the unmodified sequence.

FIG. 12 shows that chemical modification of nucleotides with PACIFIC BLUE dye produces a negative signal relative to the unmodified sequence.

FIG. 13 shows a schematic model an embodiment of the technology related to immunoreaction detection using nanopore analysis. Magnetic beads with pathogen specific antibodies bind a pathogen of interest, which then is interrogated with a dsDNA-conjugated antibody. The dsDNA contains modifications that are detected in a nanopore after melting a modified nucleic acid strand from the immunoreaction complex.

FIG. 14 shows a schematic for multiplex immunoreactions. In the schematic shown, individual pathogens have unique dsDNA-antibody probes in the schematic approach shown in FIG. 13. The probes comprise modifications that provide a distinct signature when passing through a nanopore. Thus, multiple immunoreactions are multiplexed in one assay and the counts of each pathogen are individually determined.

FIG. 15 shows a schematic of using modified probes to bind to target small RNA, ligation of double stranded products to produce a concatemer, and nanopore detection. Each “Red Tag” in the schematic has a round shape and each “Orange Tag” has a triangle shape.

FIG. 16 shows a schematic of a ligation strategy for small RNA detection.

FIG. 17 shows a schematic describing use of modified bases to resolve homopolymer stretches in a nucleic acid. Introducing a fraction of modified base during PCR (right) produces a randomly distributed signal throughout a homopolymeric stretch (right bottom) when passing through a nanopore. Thus, base calling algorithms only deal with shorter stretches of similar bases that can be resolved, rather than a continuous lack of signal from a longer region of identical bases when only unmodified bases are used (bottom left).

FIG. 18 is a table showing errors in nanopore sequencing of SNPs near homopolymer stretches. A number of non-SNP and SNP homopolymer stretches were tested with the addition of 50% modified CTP (modified with methyl, hydroxyl, hydroxymethyl, or formyl). The data indicated that methyl-CTP corrected errors in homopolymer stretches better than the other modifications.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology related to the use of modified nucleotides and nucleic acids for nucleic acid analysis (e.g., determination of base count, base composition, etc.) using a nanopore detector. Embodiments provide that modification of nucleotides is performed before, during, or after common biochemical and/or molecular biological steps such as nucleic acid amplification (e.g., by polymerase chain reaction) or reverse transcription. In some embodiments, the modification of one or more nucleotides increases or decreases the size of the nucleic acid and thus produces positive or negative changes in the electrical (e.g., current, resistance, conductance, voltage, impedance, etc.) signal as the nucleic acid passes through the nanopore. In some embodiments, the modification of one or more nucleotides in a nucleotide sequence produces detectable changes in the baseline electrical (e.g., current, resistance, conductance, voltage) signal relative to the baseline electrical (e.g., current, resistance, conductance, voltage) signal produced by the same unmodified nucleotide sequence.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, a “nanopore” refers to a pore of nanometer size (e.g., 1 to 100 nm, e.g., 1, 10, 15, 20, 25, 30, 35, 40, 45, 50, 55, 60, 65, 70, 75, 80, 85, 90, 95, or 100 nm). In some embodiments, a nanopore is a nanopore-forming protein (e.g., a hemolysin, a porin (e.g., MspA), a secretion channel (e.g., CsgG)) or a solid state nanopore in synthetic materials such as silicon or graphene. In some embodiments, the nanopore is in a lipid bilayer or in a synthetic membrane.

As used herein, a “nucleic acid” shall mean any nucleic acid molecule, including, without limitation, DNA, RNA, and hybrids thereof. The nucleic acid bases that form nucleic acid molecules can be the bases A, C, G, T and U, as well as derivatives thereof. Derivatives of these bases are well known in the art. The term should be understood to include, as equivalents, analogs of either DNA or RNA made from nucleotide analogs. The term as used herein also encompasses cDNA, that is complementary, or copy, DNA produced from an RNA template, for example by the action of a reverse transcriptase. It is well known that DNA (deoxyribonucleic acid) is a chain of nucleotides consisting of 4 types of nucleotides-A (adenine), T (thymine), C (cytosine), and G (guanine)—and that RNA (ribonucleic acid) is a chain of nucleotides consisting of 4 types of nucleotides-A, U (uracil), G, and C. It is also known that all of these 5 types of nucleotides specifically bind to one another in combinations called complementary base pairing. That is, adenine (A) pairs with thymine (T) (in the case of RNA, however, adenine (A) pairs with uracil (U)), and cytosine (C) pairs with guanine (G), so that each of these base pairs forms a double strand. The term “nucleic acid” encompasses nucleic acids that include any of the known heterocyclic bases and base analogs of DNA and RNA including, but not limited to, adenine, guanine, cytosine, thymine, uracil, inosine, xanthine, hypoxanthine, or a a heterocyclic derivative, analog, or tautomer thereof. A nucleobase can be naturally occurring or synthetic. When a nucleic acid such as an oligonucleotide is represented by a sequence of letters, such as “ATGCCTG,” it will be understood that the nucleotides are in 5′ to 3′ order from left to right and that “A” denotes deoxyadenosine, “C” denotes deoxycytidine, “G” denotes deoxyguanosine, “T” denotes thymidine, and “U” denotes uracil, unless otherwise noted. The letters A, C, G, and T may be used to refer to the bases themselves, to nucleosides, or to nucleotides comprising the bases, as is standard in the art.

As used herein, a “nucleotide” comprises a “base” (alternatively, a “nucleobase” or “nitrogenous base”), a “sugar” (in particular, a five-carbon sugar, e.g., ribose or 2-deoxyribose), and a “phosphate moiety” of one or more phosphate groups (e.g., a monophosphate, a diphosphate, or a triphosphate consisting of one, two, or three linked phosphates, respectively). Without the phosphate moiety, the nucleobase and the sugar compose a “nucleoside”. A nucleotide can thus also be called a nucleoside monophosphate or a nucleoside diphosphate or a nucleoside triphosphate, depending on the number of phosphate groups attached. The phosphate moiety is usually attached to the 5-carbon of the sugar, though some nucleotides comprise phosphate moieties attached to the 2-carbon or the 3-carbon of the sugar. Nucleotides contain either a purine (in the nucleotides adenine and guanine) or a pyrimidine base (in the nucleotides cytosine, thymine, and uracil). Ribonucleotides are nucleotides in which the sugar is ribose. Deoxyribonucleotides are nucleotides in which the sugar is deoxyribose.

In some embodiments, a nucleotide comprises a heterocyclic base (e.g., nucleobase) such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N-6-methyladenine, 2-aminoadenine, 7-deazaxanthine, 7-deazaguanine, 7- deaza-adenine, N4-ethanocytosine, 2,6-diaminopurine, N6-ethano-2,6-diaminopurine, 5-methylcytosine, 5-(C3- C6)- alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6- dihydrothymine, 5,6-dihydrouracil, 4-methyl-indole, ethenoadenine, 4 acetylcytosine, 8-hydroxy-N6-methyladenosine, aziridinylcytosine, pseudoisocytosine, 5- (carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2- dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino- methyl-2-thiouracil, beta-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, 2,6-diaminopurine, and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, La.), all herein incorporated by reference in their entireties.

Reference to a base, a nucleotide, or to another molecule may be in the singular or plural. That is, “a base” may refer to a single molecule of that base or to a plurality of the base, e.g., in a solution.

As used herein, an “abasic site” refers to a nucleotide residue in a nucleic acid that does not have a base (e.g., a purine or pyrimidine) attached to the sugar. Accordingly, in some embodiments an abasic site is an “apurinic/apyrimidinic site” or “AP site”. In some embodiments, an abasic site is produced in a synthesized nucleic acid, such as a barcode sequence, adapter, or as part of a synthesized primer or probe. In some embodiments, an abasic site is produced in a nucleic acid by incorporating uracil bases during synthesis followed by enzymatic processing with an enzyme that excises uracil bases from DNA, such as uracil-DNA glycosylase (UDG) or uracil N-glycosylase (UNG). For example, embodiments provide that uracil is incorporated into a nucleic acid using PCR and/or a probe. Treatment with UDG produces abasic sites in a known configuration within the targeted sequence and that can thus be identified.

As used herein, the term “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To illustrate, oligonucleotides are typically less than 200 residues long (e.g., between 15 and 100), however, as used herein, the term is also intended to encompass longer polynucleotide chains. Oligonucleotides are often referred to by their length. For example a 24-residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H−, NH₄+, Na+, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method. In certain embodiments, the primer is a capture primer.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification, and isolation of particular nucleic acids, gene sequences, etc.

As used herein, an “adapter” is an oligonucleotide that is linked or is designed to be linked to a nucleic acid to introduce the nucleic acid into a nanopore analysis workflow. An adapter may be single-stranded or double-stranded (e.g., a double-stranded DNA or a single-stranded DNA). As used herein, the term “adapter” refers to the adapter nucleic in a state that is not linked to another nucleic acid and in a state that is linked to a nucleic acid.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes, including but not limited to polymerase chain reaction (PCR), ligase chain reaction (LCR), helicase- dependent amplification, multiplex ligation-dependent probe amplification, real time PCR, reverse transcription PCR, nucleic acid sequence-based amplification (NASBA), and transcription-mediated amplification (TMA).

As used herein, the term “amplicon” refers to a nucleic acid generated in a nucleic acid amplification reaction, e.g., PCR and the like. As used herein, the terms “PCR product” or amplicon or “PCR fragment” generally refer to the resultant mixture of amplified DNA after two or more cycles of the PCR steps of denaturation, annealing, and extension are complete. The sequence of an amplicon includes the amplified segment of the target DNA as well as the sequence of the primers flanking the amplified region that were employed to carry out the PCR. These terms are also meant to encompass the case where there has been amplification of one or more segments of one or more target sequences.

As used herein, a “sequence” of a biopolymer refers to the order and identity of monomer units (e.g., nucleotides, amino acids, sugars, etc.) in the biopolymer. The sequence (e.g., base sequence) of a nucleic acid is typically read in the 5′ to 3′ direction.

As used herein, “nucleic acid sequence”, “nucleotide sequence”, and the like denotes any information or data that is indicative of the order of the nucleotide bases (e.g., adenine, guanine, cytosine, and thymine/uracil) in a molecule (e.g., a whole genome, a whole transcriptome, an exome, oligonucleotide, polynucleotide, fragment, etc.) of DNA or RNA.

As used herein, “moiety” refers to one of two or more parts into which something may be divided, such as, for example, the various parts of a tether, a molecule or a probe.

As used herein, a “concatemer” refers to a long continuous DNA molecule that contains multiple copies of the same DNA sequences linked in series. As an example, one possible concatemer of the nucleotide sequence ATCG is ATCGATCGATCGATCGATCG.

As used herein, a “linker” or “spacer” is a molecule or moiety that joins two molecules (e.g., nucleic acids) or moieties and provides spacing between the two molecules or moieties such that they are able to function in their intended manner. Coupling of linkers to nucleotides and substrate constructs of interest can be accomplished through the use of coupling reagents that are known in the art (see, e.g., Efimov et al., Nucleic Acids Res. 27: 4416-4426, 1999). Methods of derivatizing and coupling organic molecules are well known in the arts of organic and bioorganic chemistry. A linker may also be cleavable (e.g., photocleavable) or reversible.

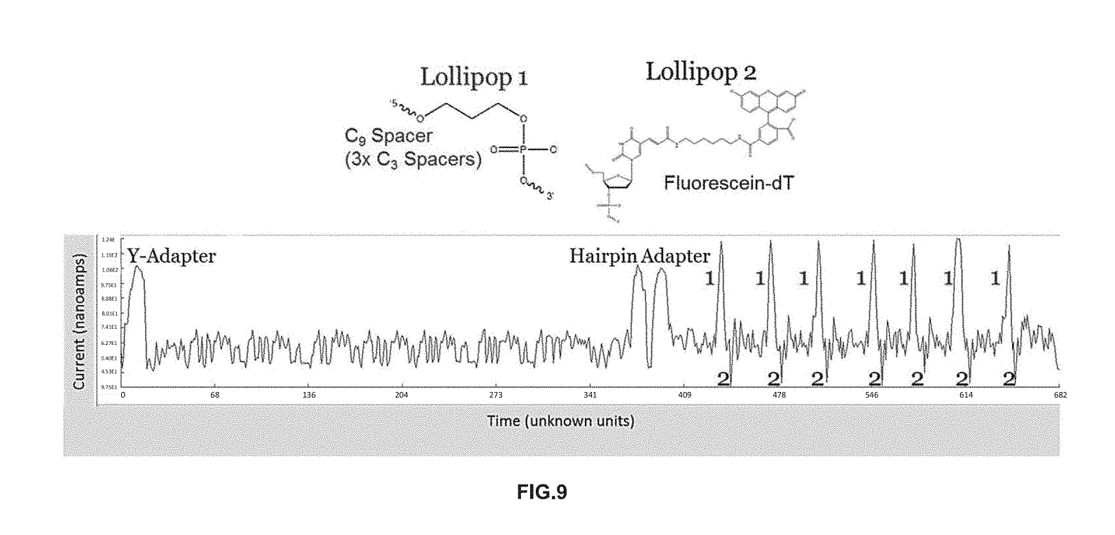

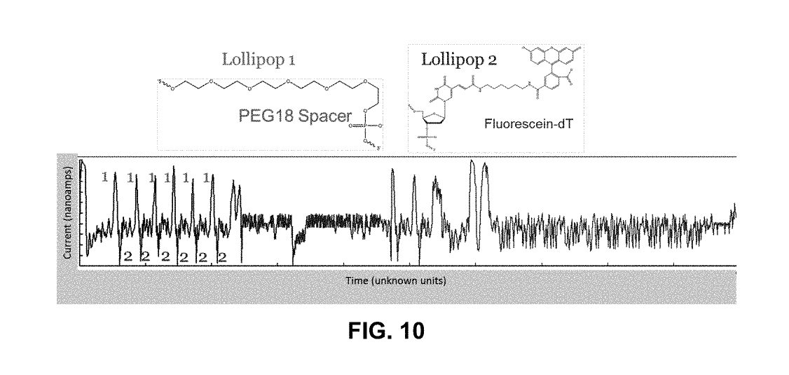

In some embodiments, a “C3 spacer” provides a connection between two other parts of a nucleic acid. In some embodiments, multiple C3 spacers are linked to provide a longer spacer, e.g., to provide a “C9” spacer comprising three C3 spacers. In some embodiments, a C3 spacer has a structure according to

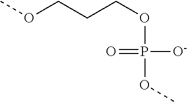

where dotted lines indicate bonds to the remainder of the nucleic acid at the 3′ and 5′ ends. See, e.g., FIG. 9. In some embodiments, a “PEG 18 spacer” provides a connection between two other parts of a nucleic acid. In some embodiments, a PEG 18 spacer has a structure according to that shown in FIG. 10 where wavy lines indicate bonds to the remainder of the nucleic acid at the 3′ and 5′ ends.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “sequencing-free” method does not comprise a sequencing step, etc.

As used herein, determining an “unordered base composition” or “unordered base count” refers to determining the base composition or base composition, respectively, of a nucleic acid without one or more of: determining a nucleotide sequence of the nucleic acid;

determining the order of any of the bases and/or nucleotides in the nucleic acid; determining the position of any base or nucleotide relative to any other base or nucleotide in the nucleic acid; determining the absolute position of any base or nucleotide in the nucleic acid; determining the position of any base or nucleotide relative to the 3′ and/or 5′ end of the nucleic acid; determining the order, relative position, or absolute position of any of the four bases A, C, G, T, or U in a nucleic acid; or determining the linear sequence of nucleotides in the nucleic acid.

### Description

As described herein, embodiments of the technology are related to incorporating one or more modified nucleotides into a nucleic acid to produce a detectable change in the nanopore current and/or conductance signal that is unique to the type of modified nucleotide introduced into the nucleic acid. In exemplary embodiments, the technology provides a signal (e.g., an electrical signal (e.g., current, resistance, conductance, voltage, impedance, etc.) measured as a function of time) used for analyzing and/or characterizing a nucleic acid, e.g., by determining base composition, base counts, presence or absence of a single nucleotide polymorphism (SNP), number of short repeat sequences, etc. Modified nucleotides can be incorporated into target sequences by many methods or may be the result of natural incorporation or chemical modification of natural bases.

Particular embodiments comprise translocating modified nucleic acid sequences through a nanopore of a nanopore detector and recording the current, conductance (inverse resistance), or other electrical variables associated with flow of background ions through or across the pore. As the nucleic acid sequence translocates through the pore, the level and duration of the electrical signal associated with the background ions varies as a function of the chemical characteristics of each nucleotide base (e.g. A, T, G, C, U) passing through the pore. The technology provided herein comprises use of modified bases to produce characteristic and detectable changes in the electrical signal. For example, in some embodiments the modified nucleotide adds to, subtracts from, and/or changes the length of the signal observed for the nucleotide to provide a new signal that is distinct for the modified nucleotide. In some embodiments, the type and/or number of the distinct and detectable current and/or conductance signatures is used for calculating base counts and/or base compositions.

Guidance for certain aspects is found in many available references and treatises well known to those with ordinary skill in the art, including, for example, Cao, Nanostructures & Nanomaterials (Imperial College Press, 2004); Levinson, Principles of Lithography, Second Edition (SPIE Press, 2005); Doering and Nishi, Editors, Handbook of Semiconductor Manufacturing Technology, Second Edition (CRC Press, 2007); Sawyer et al, Electrochemistry for Chemists, 2nd edition (Wiley Interscience, 1995); Bard and Faulkner, Electrochemical Methods: Fundamentals and Applications, 2nd edition (Wiley, 2000); Lakowicz, Principles of Fluorescence Spectroscopy, 3rd edition (Springer, 2006); Hermanson, Bioconjugate Techniques, Second Edition (Academic Press, 2008); and the like, which relevant parts are hereby incorporated by reference.

Although the disclosure herein refers to certain illustrated embodiments, it is to be understood that these embodiments are presented by way of example and not by way of limitation.

### Nanopores

The technology comprises use of a nanopore to analyze a nucleic acid. While the present technology is related to technologies for sequencing a nucleic acid by translocating the nucleic acid through the nanopore, the present technology relates to use of modified nucleic acids and/or modified nucleotides to characterize a nucleic acid (e.g., determining base count, base composition, etc.) without necessarily obtaining complete linear nucleotide sequence information, though the technology, in some embodiments, supplements sequence information obtained by nanopore sequencing. Nanopores used with various methods, systems, and devices described herein may be solid-state nanopores, protein nanopores, or hybrid nanopores comprising protein nanopores configured in a solid-state membrane, or like framework. Important features of nanopores include (i) constraining analytes, particularly polymer analytes such as a nucleic acid, to translocate through the nanopore; and (ii) compatibility with a component to translocate the nucleic acid through the nanopore, that is, whatever method is used to drive the nucleic acid through a nanopore (electrophoresis, enzyme, etc.).

A nanopore is a nanoscale pore. By nanoscale is meant that the nanopore has a diameter of less than 1 μm. In some embodiments, the nanopore has a diameter of less than 100 nm. In some embodiments, the nanopore has a diameter of 10 nm or less. Accordingly, embodiments comprise a nanopore having a size in the nanometer range (e.g., 0.1 to 1 to 10 to 100 nm, e.g., 1, 10, 15, 20, 25, 30, 35, 40, 45, 50, 55, 60, 65, 70, 75, 80, 85, 90, 95, or 100 nm). The diameter of a DNA is approximately 2 nm; thus, embodiments are provided in which the nanopore diameter is a size through which a single-stranded nucleic acid is translocated, e.g., 1 to 5 to 10 nm; e.g., approximately 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9 or 5.0 nm in diameter.

Embodiments comprise use of natural and synthetic nanopores. In some embodiments, the nanopore is a solid-state nanopore, protein nanopore, a hybrid solid state-protein nanopore, a biologically adapted solid-state nanopore, or a DNA origami nanopore. In some embodiments, the protein nanopore is alpha-hemolysin, leukocidin, Mycobacterium smegmatis porin A (MspA), outer membrane porin F (OmpF), outer membrane porin G (OmpG), outer membrane phospholipase A, Neisseria autotransporter lipoprotein (NalP), WZA, lysenin, a secretion channel (e.g., CsgG)), or a homolog or variant thereof. In some embodiments, the protein nanopore sequence is modified to contain at least one amino acid substitution, deletion, or addition. In some embodiments, the at least one amino acid substitution, deletion, or addition results in a net charge change in the nanopore. In some embodiments, the protein nanopore has a constriction zone with a non-negative charge. In another aspect, the disclosure provides a nanopore system.

For example, in some embodiments protein nanopores are precisely designed at atomic resolution using protein engineering. In some embodiments, specific modifications are designed to provide a nanopore that is a sensor for specific molecules, e.g., modified nucleic acids and/or nucleic acids comprising modified nucleotides. Engineered nanopores include nanopores that have been modified to interact with molecules passing therethrough. In some embodiments, modification of the nanopore enhances the current modulation and allows greater discrimination between different chemical modifications of the nucleic acid translocating through the nanopore.

In some embodiments, the nanopore-forming protein is embedded in an electrically resistant polymer membrane, e.g., a phospholipid bilayer, a synthetic membrane, etc. In some embodiments, the membrane is a lipid bilayer. In some embodiments, the membrane comprises a block copolymer.

In some embodiments, a nanopore is a solid state nanopore in a synthetic material (e.g., a silicon (e.g., silicon nitride (Si₃N₄), silicon dioxide (SiO₂)), graphene, alumina, titanium, gold, platinum, zirconia, or a combination thereof).

In some embodiments, the nanopore comprises a protein. A protein nanopore is a nanopore that is predominantly protein; however, other types of molecules may also be present. Examples of protein pores suitable for use in the invention include alpha hemolysin, pneumolysin, outer membrane proteins such as porins, and other bacterial pore-forming toxins (Gilbert, R. J. (2002) Cell MoI Life Sci 59, 832-44) (Parker, M. W., and Feil, S. C. (2005) Prog Biophys MoI Biol 88, 91-142) such as streptolysin O (Bhakdi, S., Tranum-Jensen, J., and Sziegoleit, A. (1985) Infect Immun 47, 52-60) or LukF (Olson, R., Nariya, H., Yokota, K., Kamio, Y., and Gouaux, E. (1999) Nat Struct Biol 6, 134-40). The latter are oligomeric assemblies of protein subunits. The diameter of the lumens of protein nanopores depends on the type of pore and ranges from 1.2 nm for alpha hemolysin (Song, L., Hobaugh, M. R., Shustak, C, Cheley, S., Bayley, H., and Gouaux, J. E. (1996) Science 274, 1859-66) to 26 nm for pneumolysin (Tilley, S. J., Orlova, E. V., Gilbert, R. J., Andrew, P. W., and Saibil, H. R. (2005) Cell 121, 247-56).

In some embodiments, the protein pore is an a-hemolysin (αHL) polypeptide. αHL is a bacterial toxin that self-assembles to form a heptameric protein pore. The X-ray structure of the aHL nanopore resembles a mushroom with a wide cap and a narrow stem, which spans the lipid bilayer (Song, L.; Hobaugh, M. R.; Shustak, C; Cheley, S.; Bayley, H.; Gouaux, J. E. Science. 1996, 274, 1859-1866). The external dimensions of the heptameric aHL pore are 10 nm×10 nm, while the central channel is 2.9 nm in diameter at the cis entrance and widens to 4.1 nm in the internal cavity. In the transmembrane region, the channel narrows to 1.3 nm at the inner constriction and broadens to 2 nm at the trans entrance of the β-barrel. The defined structure of aHL has facilitated extensive engineering studies and has led to the development of tools for the targeted permeabilization of cells (Eroglu, A.; Russo, M. J.; Bieganski, R.; Fowler, A.; Cheley, S.; Bayley, H.; Toner, M. Nat Biotechnol. 2000, 18, 163-167) as well as new biosensor elements which permit the stochastic sensing of molecules (Bayley, H.; Cremer, P. S. Nature. 2001, 413, 226-230).

Exemplary and non-limiting Staphylococcus aureus a-hemolysin wild type sequences are provided in WO2012009578 (as SEQ ID N0:20, nucleic acid coding region; SEQ ID NO:21, protein coding region, which are each incorporated herein by reference in their entireties) and available elsewhere (National Center for Bioinformatics or GenBank Accession Numbers M90536 and AAA26598). An exemplary and non-limiting Staphylococcus aureus α-hemolysin variant comprising a K131D substitution is provided as SEQ ID NO:22 in WO2012009578.

In some embodiments, the nanopore is a nucleic acid based nanopore such as those described in, e.g., EP Application Number EP20120180126 (EP Publication Number EP2695949 A1). In some embodiments, the nanopore is produced by nucleic acid origami. Methods useful in the making of DNA origami structures can be found, for example, in Rothemund 2006, Douglas et al 2009-2; Dietz et al, 2009 or U.S. Pat. No. 7,842,793 B2.

When a nanopore is immersed in a conducting fluid and a potential (voltage) is applied across the nanopore, an electric current flows due to conduction of ions through the nanopore. The current is sensitive to the size and shape of the nanopore. Translocation of nucleotides (bases), strands of DNA, or other molecules through the nanopore produces a characteristic change in the quality and/or magnitude of the current through the nanopore, the resistance across the nanopore, or the conductance across the nanopore. Monitoring the electrical signal thus provides information about the nucleotides (bases), strands of DNA, or other molecules translocating through the nanopore.

Apparatus and devices

In some embodiments, a nanopore device or apparatus comprises a material comprising one or more nanopores. A nanopore device or apparatus includes, for example, a structure comprising a first compartment (e.g., comprising a conductive liquid medium) and a second compartment (e.g., comprising the same or different conductive liquid medium) separated by a physical barrier (e.g., membrane) comprising at least one nanopore with a diameter, for example, of from about 1 to 10 nm, and that provides liquid communication between the first compartment and the second compartment; and a component for translocating the nucleic acid through the nanopore (e.g., a “translocation component”).

The technology is described in terms of a first compartment and a second compartment, but is not limited to two compartments as embodiments encompass technologies comprising a third, fourth, fifth or nth number of compartments.

In some embodiments, the translocation component applies an electric field across the barrier so that a charged molecule such as DNA passes from the first compartment through the pore to the second compartment. That is, in some embodiments the translocation component comprises a power source providing sufficient voltage to induce translocation of the nucleic acid through the nanopore, e.g., by electrophoresis or production of an electrophoretic field in which the nucleic acid is placed.

In some embodiments, the nanopore device comprises a component comprising a protein that moves the nucleic acid through the nanopore and/or controls the rate of transport the nucleic acid through the nanopore. In some embodiments, the translocation component is a molecular motor, such as a translocase, a polymerase, a helicase, an exonuclease, or a topoisomerase. In some embodiments, the polymerase is phi29 DNA polymerase, Klenow fragment, or a variant or homolog thereof. In some embodiments, the helicase is a He1308 helicase, a RecD helicase, a Tral helicase, a Tral subgroup helicase, an XPD helicase, or a variant or homolog thereof. In some embodiments, the exonuclease is exonuclease I, exonuclease III, lambda exonuclease, or a variant or homolog thereof. In some embodiments, the topoisomerase is a gyrase or a variant or homolog thereof.

The nanopore device or apparatus further comprises a component and/or system for measuring the electronic signature of a molecule passing through the nanopore.

The technology is generally described by reference to a single nanopore, but the technology comprises use of multiple nanopores, e.g., arrays of nanopores from, e.g., 10 nanopores to about 10 million nanopores. In some embodiments, arrays of 10 nanopores to 100 nanopores are used. In some embodiments, arrays of nanopores of about 100 to about 10,000 nanopores are used. In some embodiments, arrays of nanopores from about 1,000 to about 1 million nanopores are used. Arrays of nanopores are described, for example, in U.S. Pat. No. 9,017,937.

Embodiments comprise measuring an electrical signal at the nanopore as the polynucleotide moves with respect to the pore. Suitable conditions for measuring ionic currents through nanopores are known in the art and disclosed in the Examples. The technology is typically carried out with a voltage applied across the membrane and pore. The voltage used is typically from+2 V to −2 V, typically−400 mV to+400 mV. The voltage used is preferably in a range having a lower limit selected from −400 mV, −300 mV, −200 mV, −150 mV, −100 mV, −50 mV, −20 mV, and 0 mV and an upper limit independently selected from+10 mV, +20 mV, +50 mV, +100 mV, +150 mV, +200 mV, +300 mV, and+400 mV. The voltage used is more preferably in the range 100 mV to 240 mV and most preferably in the range of 120 mV to 220 mV. It is possible to increase discrimination between different nucleotides by a pore by using an increased applied potential.

Embodiments of the technology comprises use of a charge carrier, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In some embodiments, the salt is present in one or more aqueous solutions in the chambers. Potassium chloride (KCl), sodium chloride (NaCl), cesium chloride (CsCl), or a mixture of potassium ferrocyanide and potassium ferricyanide is typically used. KCl, NaCl, and a mixture of potassium ferrocyanide and potassium ferricyanide find use in some embodiments. The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M, or from 1 M to 1.4 M. In particular embodiments, the salt concentration is from 150 mM to 1 M. The technology comprises embodiments that use a salt concentration of at least 0.3 M, such as at least 0.4 M, at least 0.5 M, at least 0.6 M, at least 0.8 M, at least 1.0 M, at least 1.5 M, at least 2.0 M, at least 2.5 M, or at least 3.0 M. High salt concentrations provide a high signal to noise ratio and allow for currents indicative of the presence of a nucleotide to be identified against the background of normal current fluctuations.

The technology, in some embodiments, comprises use of a buffer. In some embodiments, the buffer is present in an aqueous solution in the chambers. Any buffer may be used in the technology. Typically, the buffer is HEPES. Another suitable buffer is Tris-HCl buffer. The technology comprises embodiments in which the pH is from 4.0 to 12.0, from 4.5 to 10.0, from 5.0 to 9.0, from 5.5 to 8.8, from 6.0 to 8.7, or from 7.0 to 8.8 or 7.5 to 8.5. In particular embodiments, the pH used is about 7.5.

The technology, in some embodiments, is performed at from 0° C. to 100° C., from 15° C. to 95° C., from 16° C. to 90° C., from 17° C. to 85° C., from 18° C. to 80° C., 19° C. to 70° C., or from 20° C. to 60° C. The methods are typically carried out at room temperature. The methods are optionally carried out at a temperature that supports enzyme function, such as about 37° C.

The apparatus is preferably configured to perform one or more methods as disclosed herein. Furthermore, embodiments provide that the nanopore apparatus comprises a sensor that performs characterization of a nucleic acid translocating through a nanopore and at least one reservoir for holding material for performing the characterization; and, in some embodiments, a fluidics system configured to controllably supply material from the at least one reservoir to the sensor device; and one or more containers for receiving respective samples, the fluidics system being configured to supply the samples selectively from the one or more containers to the sensor device. In some embodiments, the apparatus is as described in International Application No. PCT/GB08/004127, PCT/GB10/000789, PCT/GB10/002206, or PCT/US99/25679.

Nanopore devices are known in the art. See, for example, Heng, J. B. et al., The Electromechanics of DNA in a synthetic Nanopore. Biophysical Journal 2006, 90, 1098-1106; Fologea, D. et al., Detecting Single Stranded DNA with a Solid State Nanopore. Nano Letters 2005 5(10), 1905-1909; Heng, J. B. et al., Stretching DNA Using the Electric Field in a Synthetic Nanopore. Nano Letters 2005 5(10), 1883-1888; Fologea, D. et al., Slowing DNA Translocation in a Solid State Nanopore. Nano Letters 2005 5(9), 1734-1737; Bokhari, S. H. and Sauer, J. R., A Parallel Graph Decomposition Algorithm for DNA Sequencing with Nanopores. Bioinformatics 2005 21(7), 889-896; Mathe, J. et al., Nanopore Unzipping of Individual Hairpin Molecules. Biophysical Journal 2004 87, 3205-3212; Aksimentiev, A. et al., Microscopic Kinetics of DNA Translocation through Synthetic Nanopores. Biophysical Journal 2004 87, 2086-2097; Wang, H. et al., DNA heterogeneity and Phosphorylation unveiled by Single-Molecule Electrophoresis. PNAS 2004 101(37), 13472-13477; Sauer-Budge, A. F. et al., Unzipping Kinetics of Double Stranded DNA in a Nanopore. Physical Review Letters 2003 90(23), 238101-1-238101-4; Vercoutere, W. A. et al., Discrimination Among Individual Watson-Crick Base Pairs at the Termini of Single DNA Hairpin Molecules. Nucleic Acids Research 2003 31(4), 1311-131; Meller, A. et al., Single Molecule Measurements of DNA Transport Through a Nanopore. Electrophoresis 2002 23, 2583-2591. Nanopores and methods employing them are disclosed in U.S. Pat. No. 7005264 and U.S. Pat. No. 6617113 which are hereby incorporated by reference in their entireties.

The fabrication and operation of nanopores for analytical applications are disclosed in the following exemplary references that are incorporated herein by reference: Russell, U.S. Pat. No. 6,528,258; Feier, U.S. Pat. No. 4,161,690; Ling, U.S. Pat. No. 7,678,562; Hu et al, U.S. Pat. No. 7,397,232; Golovchenko et al, U.S. Pat. No. 6,464,842; Chu et al, U.S. Pat. No. 5,798,042; Sauer et al, U.S. Pat. No. 7,001,792; Su et al, U.S. Pat. No. 7,744,816; Church et al, U.S. Pat. No. 5,795,782; Bayley et al, U.S. Pat. No. 6,426,231; Akeson et al, U.S. Pat. No. 7,189,503: Bayley et al, U.S. Pat. No. 6,916,665; Akeson et al, U.S. Pat. No. 6,267,872; Meller et al, U.S. patent publication 2009/0029477; Howorka et al, International patent publication WO2009/007743; Brown et al, International patent publication WO2011/067559; Meller et al, International patent publication WO2009/020682; Polonsky et al, International patent publication WO2008/092760; Van der Zaag et al, International patent publication WO2010/007537; Yan et al, Nano Letters, 5(6): 1129-1134 (2005); Iqbal et al, Nature Nanotechnology, 2: 243-248 (2007); Wanunu et al, Nano Letters, 7(6): 1580-1585 (2007); Dekker, Nature Nanotechnology, 2: 209-215 (2007); Storm et al, Nature Materials, 2: 537-540 (2003); Wu et al, Electrophoresis, 29(13): 2754-2759 (2008); Nakane et al, Electrophoresis, 23: 2592-2601 (2002); Zhe et al, J. Micromech. Microeng., 17: 304-313 (2007); Henriquez et al, The Analyst, 129: 478-482 (2004): Jagtiani et al, J. Micromech. Microeng., 16: 1530-1539 (2006); Nakane et al, J. Phys. Condens. Matter, 15 R1365-R1393 (2003); DeBlois et al, Rev. Sci. Instruments, 41(7): 909-916 (1970); Clarke et al, Nature Nanotechnology, 4(4): 265-270 (2009); Bayley et al, U.S. patent publication 2003/0215881; and the like.

Commercial nanopore devices are available from, e.g., Oxford Nanopore

Technologies (PromethlON, MinION, SmidgION, etc.), Agilent, Sequenom, Noblegen, NABSys, and Genia.

### Modified Nucleotides and Nucleic Acids

The technology relates to modified nucleotides, modified nucleic acids, and nucleic acids comprising a modified nucleotide. Accordingly, the technology comprises use of any modification that produces a signal (or a change in a signal) when a nucleic acid comprising the modification is translocated through a nanopore. In some embodiments, the modification is a modification of a nucleotide (e.g., covalent attachment of a moiety to a nucleotide, creation of an abasic nucleotide residue, etc.). The technology is not limited in the atom of the nucleotide to which the modification is attached. In some embodiments, the nucleotide comprises a modification in the sugar; in some embodiments, the nucleotide comprises a modification in the base; in some embodiments, the nucleotide comprises a modification in the phosphate.

In some embodiments, the modification comprises modification of the structure of the nucleic acid itself (e.g., peptide nucleic acid bonds between nucleotides, linker moieties between nucleotides, phosphorothioate bonds between nucleotides, etc.). For example, the technology comprises use of the following nucleic acid and nucleotide modifications to produce detectable signals in a nanopore device.

In some embodiments, the modification is an abasic site. Abasic sites are sites in a nucleic acid that contain no base (e.g., the site is apyrimidinic or apurinic). Abasic sites are sterically hindered much less than normal nucleotides and thus are contemplated to behave differently than normal nucleotides when translocating through a nanopore. Abasic sites occur spontaneously (e.g., by spontaneous hydrolysis of the N-glycosylic bond), or under the action of radiation or alkylating agents, or enzymatically as an intermediate in the repair of modified or abnormal bases (see below). In some embodiments, abasic sites are introduced into a nucleic acid during synthesis of the nucleic acid (e.g., by introduction of an abasic nucleotide residue into the polymer) or after the synthesis of the nucleic acid (e.g., by cleavage of the base from the nucleic acid by hydrolysis of the Nglycosylic bond). In some embodiments, an abasic site is produced at a site where a DNA comprises a uracil. Uracil in DNA can be produced by the deamination of cytosine. The presence of uracil in DNA constitutes a mutation. Accordingly, enzymes exist to remove uracil from DNA. For example, uracil-DNA Glycosylase (UDG) is an enzyme that excises uracil from single-stranded DNA or double stranded-DNA by hydrolyzing the glycosidic bond to produce an abasic site as a first step in correcting the presence of uracil in DNA.

Additional information on glycosylase mechanisms and structures is provided in the art, e.g., in A. K. McCullough, et al., Annual Rev of Biochem 1999, 68, 255. In particular, four DNA glycosylases (ROS1, DME, DML2, and DML3) have been identified in Arabidopsis thaliana that remove methylated cytosine from double-stranded DNA, leaving an abasic site. (See, e.g., S. K. Ooi, et al., Cell 2008, 133, 1145, incorporated herein by reference in its entirety for all purposes.)

In some embodiments, nucleic acids comprise a DNA spacer. DNA spacers are chemical modifications of a nucleic acid that create distance between two nucleic acid segments in a sequence. For example, DNA spacers include but are not limited to commercially available spacers based on phosphoramidite, hexanediol, triethylene glycol, and hexa-ethyleneglycol. The addition of a DNA spacer to a nucleic acid creates a gap in the nucleotide sequence that changes the interaction of the nucleic acid with the nanopore and changes (e.g., increases) the conductance of the nanopore, which produces a change in the signal data. Spacers are available having different lengths (e.g., in some embodiments, spacers produce a distance between consecutive nucleotides that is smaller than the natural linkage between nucleotides; in some embodiments, spacers produce a distance between consecutive nucleotides that is equal to or greater than the natural linkage between nucleotides). During translocation of the nucleic acid through the nanopore, the signal provides temporal information (e.g., time domain) in addition to the quality and magnitude of the electrical (e.g., conductance, current, resistance, voltage, impedance, etc.) signal. Thus, spacers of different lengths produce distinct temporal signatures in the data, e.g., for combinatorial analysis. In some embodiments, the spacer comprises one or more C3 spacers. In some embodiments, the spacer comprises a PEG (e.g., a PEG18).

In some embodiments, nucleic acids comprise a nucleotide modified chemically (e.g., in some embodiments nucleic acids comprise a chemically-modified nucleotide, e.g., in some embodiments nucleic acids comprise a nucleotide comprising a chemically-modified base, sugar, or phosphate). For instance, chemicals such as biotin, azide, glucose, and EDTA are added to a nucleotide base to create a modified nucleotide detectable by a nanopore.

In some embodiments, the technology comprises use of drag tags or parachute primers. For example, embodiments provide that nucleic acids are synthesized or modified to include a tag that slows passage of the nucleic acid through a nanopore. Drag tags include, but are not limited to, chemical modifications that slow passage of a nucleic acid by steric or electric interference and/or nucleic acid modifications that form a steric “parachute” to slow passage through a pore. Because nanopore detection includes both electrical signal information (e.g., current, conductance, resistance, voltage) and time of passage through the pore, slowing the speed that a nucleic acid translocates through a nanopore provides a signature in the data than can be identified and characterized using a less complicated analysis than determining the full nucleotide sequence.

In some embodiments, nucleic acids are treated with a lambda exonuclease to remove a non-derivatized strand. Lambda exonuclease is an enzyme that digests DNA in a 5′ to 3′ orientation and that is vastly accelerated by the presence of a 5′ phosphate group. Lambda exonuclease does not digest DNA at gaps, so after creation of abasic sites or DNA linkers, lambda exonuclease digests only the unaltered strand. Accordingly, in some embodiments, treatment with lambda exonuclease provides a method of reducing sample complexity and enhancing the signal of modified nucleic acids.

In some embodiments, nucleic acids comprise amino modified nucleotides. Amino modified nucleotides, or aminoallyl nucleotides, are modified nucleotides that contain an allylamine In some embodiments, the allylamine group is further modified post-synthesis, e.g., by conjugating a moiety to the allylamine group, e.g., a fluorescent moiety. Thus, in some embodiments, post-synthesis conjugation of moieties to nucleic acids provides a technology in which modifications are added to a nucleic acid to provide analysis (e.g., and read out) by a nanopore device. In addition, in some embodiments the allylamine group is added during synthesis or amplification (e.g., PCR) or included as a probe for a specific target. Embodiments provide that the number and spacing of allylamines is altered to produce distinctly identifiable signals when passing through a nanopore.

In some embodiments, nucleic acids comprise a quantum dot. Quantum dots are semiconductors that have tunable size and shape and tunable electric and optical properties. In some embodiments, a nucleic acid comprising a quantum dot is sterically hindered when passing through a nanopore. Embodiments relate to the use of various materials to produce quantum dots that have tunable and distinct conductive properties that are detectable and/or distinguishable as they pass through a nanopore. Most quantum dots are contemplated to be too large for passage through protein-based nanopores but quantum dots find use in solid-state nanopore systems. In some embodiments, both the quantum dot and solid-state nanopore (e.g., nanopore size) are tunable and thus the quantum dot and nanopore can be tuned for appropriate (e.g., optimal) interact with one another, e.g., to produce a robust signal to provide information about the nucleic acid.

In some embodiments, nucleic acids comprise a hairpin or a circular nucleic acid (e.g., DNA, RNA). Nucleic acids containing hairpins or circular nucleic acid produce steric and/or temporal effects. Embodiments provide that the size of the hairpin or circular nucleic acid is modifiable to produce unique electrical and temporal signatures as they pass through a nanopore.

In some embodiments, nucleic acids comprise double labeling (e.g., nucleic acids comprise a modification (e.g., as described herein) on both strands of a double stranded product). For example, modifying both strands of a double-stranded nucleic acid (e.g., modifying one or more nucleotides on each strand of a double-stranded nucleic acid) provides, in some embodiments, a distinct signature when the nucleic acid is translocated through a nanopore. In embodiments of the technology, the two strands of a double-stranded nucleic acid are covalently connected by a hairpin adaptor on one end of the double-stranded nucleic acid. Conversion to a single-stranded form thus provides a single-stranded nucleic acid comprising both strands of the original duplex. When translocated through a nanopore, the two strands are translocated in series as part of one nucleic acid. For example, embodiments provide use of a single probe directed against many distinctly labeled targets to produce distinct or unique signatures for every target.

In some embodiments, a nucleic acid is modified, derivatized, and/or bound to an antibody or other protein. Proteins provide a large steric signal, or a temporal blockade of signal, when passing through a nanopore. Examples of potential targets of antibodies or other proteins are, without limitation, antibodies against epigenomic targets like DNA methylation or transcription factors that can identify unique sequences.

In some embodiments, nucleic acids comprise a coordination complex. Coordination complexes are metal-containing structures that bind nucleic acids. The technology thus contemplates embodiments in which nucleic acids comprise coordination complexes that fit in a groove of the nucleic acid with sequence specificity. In some embodiments, the technology comprises a coordination complex that is conjugated to a nucleic acid probe that binds to a target of interest. The metals in the coordination complexes provide a distinct signature when passing through a nanopore.

In some embodiments, a nucleic acid comprises one or more portions that are uncharged. For example, some embodiments relate to a nucleic acid comprising peptide nucleic acid (PNA). Peptide nucleic acids (PNA) are artificially designed nucleic acid-like molecules without a phosphate backbone. In particular, the PNA backbone comprises repeating N-(2-aminoethyl)-glycine units linked by peptide bonds. The purine and pyrimidine bases are linked to the backbone by a methylene bridge and a carbonyl group. Because PNA do not contain a charged phosphate background, their passage through a nanopore produces a much different signal than DNA or RNA. Additionally, PNA bind to complementary nucleic acids stronger than nucleic acids in probe hybridization and thus provide a method for producing shorter probe molecules.

In some embodiments, nucleic acids comprise a dendrimer, e.g., a dendrimer comprising a tag or modification on one or both branch(es) of the dendrimer. DNA dendrimers are branched units containing many DNA units that can contain different DNA sequences. Embodiments provide that the branches of a DNA dendrimer comprise any of the modifications described herein. Further, embodiments provide that the dendrimers are conjugated or hybridized to a target of interest to produce a distinct signal. In some embodiments, dendrimers are modified to comprise multiple modifications. In some embodiments, dendrimer size is modified to affect translocation of the nucleic acid through a pore, thus providing a technology for combinatorial tagging.

In some embodiments, the modifications described herein are introduced into primers and/or probes. In some embodiments, the modifications described herein are introduced into primers and/or probes to produce barcoded samples for downstream nucleic acid detection. That is, in some embodiments the technology comprises use of a primer comprising a modification that is detectable by a nanopore; in some embodiments the technology comprises use of a plurality of primers comprising modifications that are detectable and/or distinguishable by a nanopore and/or by analysis of data collected from a nanopore device. In some embodiments, the technology comprises use of a probe comprising a modification that is detectable by a nanopore; in some embodiments the technology comprises use of a plurality of probes comprising modifications that are detectable by a nanopore.

In some embodiments, amplification primers (e.g., for polymerase chain reaction, linear chain reaction, reverse transcription polymerase chain reaction, real-time polymerase chain reaction, etc.) are modified according to the technology provided herein, e.g., by a modification that is detectable by a nanopore. In some embodiments, a first primer (e.g., a forward primer; a reverse primer) comprises a nucleic acid or nucleotide modification according to the technology provided herein. In some embodiments, a second primer (e.g., a reverse primer; a forward primer) comprises the same nucleic acid or nucleotide modification as the first primer. In some embodiments, a second primer (e.g., a reverse primer; a forward primer) comprises a different nucleic acid or nucleotide modification as the first primer. Embodiments provide technologies comprising 1, 2, 3, 4, 5, 6, 7, 8, 9, 10, 11, 12, 13, 14, 15, 16, 17, 18, 19, 20 or more primers, each of which comprises a modification that, in some embodiments, is the same as one or more modifications of one or more other primers and/or that, in some embodiments, is different from one or more modifications of one or more other primers. As an illustrative and non-limiting example, in some embodiments a first primer comprises a first barcode and a second primer comprises the same first barcode or a second barcode. The barcodes provide for detection of an amplicon produced in an amplification reaction by the first and second primers, e.g., for detection of the amplicon moving through a nanopore. Further illustrating this non-limiting example, a third primer comprises a second barcode or a third barcode and a fourth primer comprises the same barcode or a fourth barcode. The barcodes provide for detection of a second amplicon produced in an amplification reaction by the third and fourth primers, e.g., for detection of the second amplicon moving through a nanopore and/or differentiation of the second amplicon from the first amplicon.

In some embodiments, modified nucleic acids are produced by an amplification reaction (e.g., polymerase chain reaction, linear chain reaction, reverse transcription polymerase chain reaction, real-time polymerase chain reaction, etc.). In some embodiments, a modified nucleotide is introduced into a nucleic acid by an amplification reaction (e.g., an amplification reaction is performed with one or more modified nucleotide(s) that is/are incorporated into the amplicon during the synthesis step(s) of the amplification reaction). In some embodiments, a precursor of a modified nucleotide is introduced into a nucleic acid by an amplification reaction (e.g., an amplification reaction is performed with one or more precursors of a modified nucleotide that is/are incorporated into the amplicon during the extension (e.g., synthesis) step(s) of the amplification reaction). Then, in some embodiments, the modified nucleotide is produced in the nucleic acid by a chemical reaction that converts the precursor of a modified nucleotide to a modified nucleotide.

In some embodiments, the modifications are catalogued in a centralized database that provides information about the electrical signal (e.g., conductance, current, resistance, voltage, impedance, etc.) and temporal signal (e.g., changes in electrical and/or temporal) signal caused by each individual modification. In addition, embodiments provide that modifications are used combinatorially to provide a library of different signatures that are used to tag individual base sequences or individual samples within a pool.

In some embodiments, a nucleic acid and/or a nucleotide is modified with a fluorescent moiety (e.g., a fluorogenic dye, also referred to as a “fluorophore” or a “fluor”). A wide variety of fluorescent moieties is known in the art and methods are known for linking a fluorescent moiety to a nucleotide prior to incorporation of the nucleotide into an oligonucleotide and for adding a fluorescent moiety to an oligonucleotide after synthesis of the oligonucleotide.

Examples of compounds that may be used to modify a nucleotide and/or a nucleic acid include but are not limited to xanthene, anthracene, cyanine, porphyrin, and coumarin dyes, e.g., xanthene derivatives such as fluorescein, rhodamine, Oregon green, eosin, and TEXAS RED dye; cyanine derivatives such as cyanine, indocarbocyanine, oxacarbocyanine, thiacarbocyanine, and merocyanine; naphthalene derivatives (dansyl and prodan derivatives); coumarin derivatives; oxadiazole derivatives such as pyridyloxazole, nitrobenzoxadiazole, and benzoxadiazole; pyrene derivatives such as cascade blue; oxazine derivatives such as Nile red, Nile blue, cresyl violet, and oxazine 170; acridine derivatives such as proflavin, acridine orange, and acridine yellow; arylmethine derivatives such as auramine, crystal violet, and malachite green; and tetrapyrrole derivatives such as porphin, phtalocyanine, bilirubin.

Examples of xanthene dyes that find use with the present technology include but are not limited to fluorescein, 6-carboxyfluorescein (6-FAM dye), 5-carboxyfluorescein (5-FAM dye), 5- or 6-carboxy-4,7,2′,7′-tetrachlorofluorescein (TET dye), 5- or 6-carboxy-4′5′2′4′5′7′ hexachlorofluorescein (HEX dye), 5′ or 6′-carboxy-4′,5′-dichloro-2,′7′-dimethoxyfluorescein (JOE dye), 5-carboxy-2′,4′,5′,7′-tetrachlorofluorescein (ZOE dye), rhodol, rhodamine, tetramethylrhodamine (TAMRA dye), 4,7- dlchlorotetramethyl rhodamine (DTAMRA dye), rhodamine X (ROX dye), and TEXAS RED dye. Examples of cyanine dyes that may find use with the present invention include but are not limited to CY3 dye, CY3B dye, CY3.5 dye, CY5 dye, CY5.5 dye, CY7 dye, and CY7.5 dye. Other fluorescent moieties and/or dyes that find use with the present technology include but are not limited to energy transfer dyes, composite dyes, and other aromatic compounds that give fluorescent signals. In some embodiments, the fluorescent moiety comprises a quantum dot.

Additional examples of compounds that may be used to modify a nucleotide and/or a nucleic acid include but are not limited to, d-Rhodamine acceptor dyes including CY5 dye, dichloro[R110,] dichloro[R6G], dichloro[TAMRA], dichloro[ROX] or the like, fluorescein donor dyes including fluorescein, 6-FAM, 5-FAM, or the like; Acridine including Acridine orange, Acridine yellow, Proflavin, pH 7, or the like; Aromatic Hydrocarbons including 2-Methylbenzoxazole, Ethyl p-dimethylaminobenzoate, Phenol, Pyrrole, benzene, toluene, or the like; Arylmethine Dyes including Auramine O, Crystal violet, Crystal violet, glycerol, Malachite Green or the like; Coumarin dyes including 7-Methoxycoumarin-4-acetic acid, Coumarin 1, Coumarin 30, Coumarin 314, Coumarin 343, Coumarin 6 or the like; Cyanine Dyes including 1,1′-diethyl-2,2′-cyanine iodide, Cryptocyanine, Indocarbocyanine (C3) dye, Indodicarbocyanine (C5) dye, Indotricarbocyanine (C7) dye, Oxacarbocyanine (C3) dye, Oxadicarbocyanine (C5) dye, Oxatricarbocyanine (C7) dye, Pinacyanol iodide, Stains all, Thiacarbocyanine (C3) dye, ethanol, Thiacarbocyanine (C3) dye, n-propanol, Thiadicarbocyanine (C5) dye, Thiatricarbocyanine (C7) dye, or the like; Dipyrrin dyes including N,N′-Difluoroboryl-1,9-dimethyl-5-(4-iodophenyl)-dipyrrin, N,N′-Difluoroboryl-1,9- dimethyl-5-[(4-(2-trimethylsilylethynyl), N,N′-Difluoroboryl- 1,9- dimethyl- 5-phenydipyrrin, or the like; Merocyanines including 4-(dicyanomethylene)-2-methyl-6-(p-dimethylaminostyryl)-4H-pyran (DCM), acetonitrile, 4-(dicyanomethylene)-2-methyl-6-(p-dimethylaminostyryl)-4H-pyran (DCM), methanol, 4-Dimethylamino-4′-nitrostilbene, Merocyanine 540, or the like; Miscellaneous Dyes including 4′,6-Diamidino-2-phenylindole (DAPI), dimethylsulfoxide, 7-Benzylamino-4-nitrobenz-2-oxa-1,3-diazole, Dansyl glycine, Dansyl glycine, dioxane, Hoechst 33258, DMF, Hoechst 33258, Lucifer yellow CH, Piroxicam, Quinine sulfate, Quinine sulfate, Squarylium dye III, or the like; Oligophenylenes including 2,5-Diphenyloxazole (PPO), Biphenyl, POPOP, p-Quaterphenyl, p-Terphenyl, or the like; Oxazines including Cresyl violet perchlorate, Nile Blue, methanol, Nile Red, ethanol, Oxazine 1, Oxazine 170, or the like; Polycyclic Aromatic Hydrocarbons including 9,10-Bis(phenylethynyl)anthracene, 9,10- Diphenylanthracene, Anthracene, Naphthalene, Perylene, Pyrene, or the like; polyene/polyynes including 1,2-diphenylacetylene, 1,4-diphenylbutadiene, 1,4-diphenylbutadiyne, 1, 6-Diphenylhexatriene, Beta-carotene, Stilbene, or the like; Redox-active Chromophores including Anthraquinone, Azobenzene, Benzoquinone, Ferrocene, Riboflavin, Tris(2,2′-bipyridypruthenium(II), Tetrapyrrole, Bilirubin, Chlorophyll a, diethyl ether, Chlorophyll a, methanol, Chlorophyll b, Diprotonated-tetraphenylporphyrin, Hematin, Magnesium octaethylporphyrin, Magnesium octaethylporphyrin (MgOEP), Magnesium phthalocyanine (MgPc), PrOH, Magnesium phthalocyanine (MgPc), pyridine, Magnesium tetramesitylporphyrin (MgTMP), Magnesium tetraphenylporphyrin (MgTPP), Octaethylporphyrin, Phthalocyanine (Pc), Porphin, ROX dye, TAMRA dye, Tetra-t-butylazaporphine, Tetra-t-butylnaphthalocyanine, Tetrakis(2,6-dichlorophenyl)porphyrin, Tetrakis(o-aminophenyl)porphyrin, Tetramesitylporphyrin (TMP), Tetraphenylporphyrin (TPP), Vitamin B12, Zinc octaethylporphyrin (ZnOEP), Zinc phthalocyanine (ZnPc), pyridine, Zinc tetramesitylporphyrin (ZnTMP), Zinc tetramesitylporphyrin radical cation, Zinc tetraphenylporphyrin (ZnTPP), or the like; Xanthenes including Eosin Y, Fluorescein, basic ethanol, Fluorescein, ethanol, Rhodamine 123, Rhodamine 6G, Rhodamine B, Rose bengal, Sulforhodamine 101, or the like; PACIFIC BLUE dye, PACIFIC ORANGE dye, PACIFIC GREEN dye, or the like; or mixtures or combination thereof or synthetic derivatives thereof.

Further examples of compounds that may be used to modify a nucleotide and/or a nucleic acid include but are not limited to a fluorescent moiety that is xanthene, fluorescein, rhodamine, BODIPY, cyanine, coumarin, pyrene, phthalocyanine, phycobiliprotein, ALEXA FLUOR® 350, ALEXA FLUOR® 405, ALEXA FLUOR® 430, ALEXA FLUOR® 488, ALEXA FLUOR® 514, ALEXA FLUOR® 532, ALEXA FLUOR® 546, ALEXA FLUOR® 555, ALEXA FLUOR® 568, ALEXA FLUOR® 568, ALEXA FLUOR® 594, ALEXA FLUOR® 610, ALEXA FLUOR® 633, ALEXA FLUOR® 647, ALEXA FLUOR® 660, ALEXA FLUOR® 680, ALEXA FLUOR® 700, ALEXA FLUOR® 750, or a squaraine dye. In some embodiments, a nucleotide and/or a nucleic acid is modified with a fluorescently detectable moiety as described in, e.g., Haugland (September 2005) MOLECULAR PROBES HANDBOOK OF FLUORESCENT PROBES AND RESEARCH CHEMICALS (10th ed.), which is herein incorporated by reference in its entirety.

In some embodiments a nucleic acid and/or nucleotide is modified with a moiety available from ATTO-TEC GmbH (Am Eichenhang 50, 57076 Siegen, Germany), e.g., as described in U.S. Pat. Appl. Pub. Nos. 20110223677, 20110190486, 20110172420, 20060179585, and 20030003486; and in U.S. Pat. No. 7,935,822, all of which are incorporated herein by reference (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO740).

In embodiments comprising modified nucleotides, the modification is linked to the nucleotide. The technology is not limited in how this link is produced. In some embodiments, the modification is attached to the nucleotide by a covalent linkage such as an amide bond, disulfide bond, thioether bond, or a linkage generated by a Diels-Alder reaction, click chemistry, or related pericyclic reactions. In some embodiments, purine bases comprise a chemical linker at position 7; in some embodiments, pyrimidine bases comprise a chemical linker at position 5. These positions are preferred in some embodiments because nucleotide analogs comprising linkers at these positions are known to be accepted as substrates by polymerase enzymes.

The modifications are attached at various stages of the synthesis of a nucleic acid. For example, in some embodiments synthetic oligonucleotides that are used as primers are obtained by linking the modification to a synthetic oligonucleotide carrying a modified base with a linker. Alternatively, in some embodiments oligonucleotides comprising modifications are obtained by solid phase oligonucleotide synthesis using phosphoramidite analogs carrying the modification. Furthermore, in some embodiments the modifications are incorporated into a DNA strand by template-directed DNA polymerization using triphosphate nucleotide analogues. In some embodiments, the polymerization is performed using nucleotide analogs comprising the modification. In embodiments in which the polymerase does not incorporate modified nucleotides the technology uses nucleotides comprising a linker, which are subsequently derivatized with the modification after the base has been incorporated into the DNA strand. In some embodiments, incorporation of modified nucleotides comprises use of a “promiscuous” polymerase such as Deep Vent exo-(JACS, 2006, 128, 1398-1399).

In some embodiments, a nucleic acid is exposed to a reagent that transforms a modified nucleotide to a different nucleotide structure. For example, a bacterial cytosine methyl transferase converts 5-methylcytosine to thymine (M. J. Yebra, et al., Biochemistry 1995, 34(45), 14752, incorporated herein by reference in its entirety for all purposes). Alternatively, in some embodiments the reagent converts a methyl-cytosine to 5-hydroxy-methylcytosine, e.g., the hydroxylase enzyme TET1 (M. Tahiliani, et al., Science 2009, 324(5929), 930, incorporated herein by reference in its entirety for all purposes). In further embodiments, the reagent comprises a cytidine deaminase activity that converts methyl-cytosine to thymine (H. D. Morgan, et al., J Biological Chem 2004, 279, 52353, incorporated herein by reference in its entirety for all purposes).

### Methods

Embodiments of the technology relate to characterizing a nucleic acid by translocating the nucleic acid through a nanopore and monitoring an electrical signal describing current, resistance, conductance, and/or impedance at the nanopore. Embodiments provide methods comprising steps of preparing a sample, analyzing a nucleic acid with a nanopore (e.g., translocating a nucleic acid through a nanopore, e.g., a nanopore of a nanopore device), and collecting data (e.g., comprising an electrical signal (e.g., current, impedance, resistance, conductance) measured as a function of time), and analyzing data (e.g., using bioinformatics, statistics, signal processing, etc.), e.g., in some embodiments without necessarily acquiring nucleotide sequence data, though, in some embodiments the present technology supplements and/or improves nucleic acid sequencing.

Some embodiments comprise providing a nanopore device or apparatus comprising a first compartment and a second compartment separated by a physical barrier (e.g., membrane) comprising at least one nanopore with a diameter. Some embodiments comprise providing a sample comprising a nucleic acid.

The method comprises translocating a nucleic acid through a nanopore from a first compartment (e.g., comprising a conductive liquid medium) to a second compartment (e.g., comprising the same or different conductive liquid medium), wherein the nanopore is disposed in a physical barrier (e.g., a membrane) and provides liquid communication between the first compartment and the second compartment.

In some embodiments, methods further comprise applying an electrical potential between the first compartment and the second compartment to translocate the nucleic acid through the nanopore. In some embodiments, the methods further comprise providing a translocation component (e.g., a protein that mechanically drives the nucleic acid through the nanopore) to translocate the nucleic acid through the nanopore.

In some embodiments, methods comprise measuring electrical signals (e.g., current, conductance, impedance, resistance, tunneling (Ivanov A P et al., Nano Lett. 2011 Jan. 12; 11(1)279-85), and field effect transistor measurements (International Application WO 2005/124888)) at the nanopore as the nucleic acid translocates through the nanopore. In some embodiments, the method also comprises identifying a subset of the plurality of measured electrical signals associated with a single translocation step of the nucleic acid. In some embodiments, the method also comprises determining a characteristic of the nucleic acid based on the electrical signals.

Electrical measurements may be made using standard single channel recording equipment as describe in Stoddart D et al., Proc Natl Acad Sci, 12; 106(19)7702-7, Lieberman K Ret al, J Am Chem Soc. 2010; 132(50)17961-72, and International Application WO-2000/28312. Alternatively, electrical measurements may be made using a multi-channel system, for example as described in International Application WO-2009/077734 and International Application WO-2011/067559.

In some embodiments, the technology provides a method for analyzing a nucleic acid in a nanopore system, as described herein. Embodiments of the method comprise translocating the nucleic acid through the nanopore from the first compartment to the second compartment; applying an electrical potential between the first compartment and the second compartment as the nucleic acid translocates through the nanopore; measuring a plurality of electrical signals between the first compartment and the second compartment as the nucleic acid is in or translocates through in the nanopore; and determining a characteristic of the nucleic acid based on the measured current signals.

In some embodiments, data are analyzed. Analysis of the data generated by the technology described herein is generally performed using software and/or statistical algorithms that perform various data conversions, e.g., conversion of electrical signals measured as a function of time to base composition, base number, repeat number, etc. Such software, statistical algorithms, and use thereof are described in detail, e.g., in U.S. Patent Publication No. 20090024331 and U.S. pat. app. Ser. Nos. 12/592,284 and 13/731,506, the disclosure of each of which is incorporated herein by reference in its entirety for all purposes. Specific methods for discerning altered nucleotides in a template nucleic acid are provided in U.S. pat. app. Ser. Nos. 12/635,618, 12/945,767, 13/633,673, 13/930,178, and 14/863,133, each of which is incorporated herein by reference in its entirety for all purposes. These methods include use of statistical classification algorithms that analyze the signal from a single-molecule sequencing technology (e.g., a nanopore device) and detect changes in one or more aspects of signal morphology, variation of reaction conditions, and adjustment of data collection parameters to increase sensitivity to changes in signal due to the presence of modifications in nucleic acids.

Some embodiments comprise detecting a change in the amplitude, frequency, or shape of the electrical signal (e.g., an electrical signal measured as a function of time).

In some embodiments, the technology provides methods for detecting changes in the kinetics (e.g., slowing, speeding, pausing, etc.) or other reaction data for translocation of a nucleic acid through a nanopore. It is appreciated that the kinetic activity of single molecules does not follow the regular and simple picture implied by traditional chemical kinetics, a view dominated by single-rate exponentials and the smooth results of ensemble averaging. See, e.g., Herbert, et al. (2008) Ann Rev Biochem 77: 149. As such, methods are provided to analyze the data generated for single nucleic acids. General information on algorithms for use in related technologies for sequence analysis can be found, e.g., in Braun, et al. (1998) Statist Sci 13: 142; and Durbin, et al. (1998) Biological sequence analysis: Probabilistic models of proteins and nucleic acids, Cambridge University Press: Cambridge, UK.

For example, in some embodiments methods comprise detecting repeat sequences in a nucleic acid. In some embodiments, the technology comprises modifying a nucleotide in each instance of a repeat sequence, e.g., linking the nucleotide to a moiety that produces a detectable signal when the nucleic acid is translocated through a nanopore, creating an abasic site at the nucleotide, and/or using another modification as described herein.

In some embodiments, methods comprise ligating a nucleic acid to a nucleic acid to be analyzed. In some embodiments, methods comprise ligating an adapter to the nucleic acid to be analyzed (e.g., a hairpin adapter, an adapter for promoting translocation of the nucleic acid through the nanopore, an adapter comprising a barcode). In some embodiments, methods comprise ligating a nucleic acid to a nucleic acid to be analyzed to provide a nucleic acid of sufficient length for translocation of the nucleic acid through the nanopore. In some embodiments, a nucleic acid to be analyzed in processed with a commercial library preparation kit.

Some embodiments comprise identifying an electrical signal associated with a modified nucleotide in a nucleic acid, e.g., an electrical signal produced by the modified nucleotide when passing through a nanopore.

Some embodiments comprise producing an abasic site in a nucleic acid, e.g., by incorporation of uracil in a DNA followed by excising the uracil base (e.g., using an enzyme that excises uracil bases from DNA, such as uracil-DNA glycosylase (UDG) or uracil N-glycosylase (UNG). Related embodiments comprise deaminating cytosine to produce uracil in a DNA followed by excising the uracil base (e.g., using an enzyme that excises uracil bases from DNA, such as uracil-DNA glycosylase (UDG) or uracil N-glycosylase (UNG).

Some embodiments comprise identifying formation of an immunocomplex, e.g., a complex formed by an antigen and an antigen-recognizing molecule that specifically recognizes and binds to the antigen (e.g., antibody, antibody fragment, etc.)

Some embodiments comprise identifying a small RNA in a sample. In some embodiments, identifying a small RNA comprises hybridizing a probe to an RNA in a sample.

### Systems

Some embodiments are related to systems for analysis of a nucleic acid, e.g., characterizing a nucleic acid (e.g., with respect to base count, base composition, sequence repeats, methylation status, etc.) without necessarily determining a nucleotide sequence, though embodiments relate to providing characterization of a nucleic acid that supplements a nucleotide sequence. For example, in some embodiments, the technology provides systems comprising a nanopore analysis device (e.g., a nanopore sequencer) and a modified nucleotide or a plurality of modified nucleotides. In some embodiments, the technology provides systems comprising a nanopore analysis device (e.g., a nanopore sequencer) and a reagent for producing a modified nucleotide or a plurality of modified nucleotides (e.g., an enzyme, an enzyme and one or more substrates, a chemical moiety, a chemical moiety comprising a reactive group for attachment to a nucleic acid and/or a nucleotide).

In some embodiments, systems comprise the components of a membrane, such as the phospholipids needed to form an amphiphilic layer, such as a lipid bilayer. In some embodiments, systems comprise one or more other reagents or instruments to enable any of the embodiments mentioned above to be carried out. Such reagents or instruments include one or more of the following: suitable buffer(s) (aqueous solutions), components to obtain a sample from a subject (such as a vessel or an instrument comprising a needle), reagents to amplify and/or express polynucleotides, a membrane as defined above or a voltage or patch clamp apparatus. Reagents may be present in a dry state such that a fluid sample resuspends the reagents. The system may, optionally, comprise nucleotides.

Some system embodiments of the technology provided herein further comprise functionalities for collecting, storing, and/or analyzing data. For example, in some embodiments the device comprises a processor, a memory, and/or a database for, e.g., storing and executing instructions, analyzing data, performing calculations using the data, transforming the data, and storing the data. Moreover, in some embodiments a processor is configured to control the device. In some embodiments, the processor is used to initiate and/or terminate the measurement and data collection. In some embodiments, the device comprises a user interface (e.g., a keyboard, buttons, dials, switches, and the like) for receiving user input that is used by the processor to direct a measurement. In some embodiments, the device further comprises a data output for transmitting data to an external destination, e.g., a computer, a display, a network, and/or an external storage medium. Some embodiments provide that the device is a small, handheld, portable device incorporating these features and components.

Some embodiments comprise a networked cluster of nanopore analysis devices and a computer, e.g., to control the individual devices of the cluster and to accept and process data from the cluster.

Some embodiments comprise a remote computer for processing data, e.g., data that is transmitted from one or more nanopore devices and/or data that is transmitted from one or more clusters of nanopore devices.

### Kits

Some embodiments relate to kits for analysis of a nucleic acid, e.g., characterizing a nucleic acid (e.g., with respect to base count, base composition, sequence repeats, methylation status, etc.) without necessarily determining a nucleotide sequence, though embodiments relate to providing characterization of a nucleic acid that supplements a nucleotide sequence. In some embodiments, kits comprise reagents for preparing a nucleic acid for analysis on a nanopore analysis device (e.g., a commercial nanopore sequencer). For example, kit embodiments comprise one or more of: a modified nucleotide or a plurality of modified nucleotides; a reagent for producing a modified nucleotide or a plurality of modified nucleotides (e.g., an enzyme, an enzyme and one or more substrates, a chemical moiety, a chemical moiety comprising a reactive group for attachment to a nucleic acid and/or a nucleotide); adapters (e.g., a hairpin adaptor; an adapter that directs a nucleic acid to a nanopore for translocation and analysis); a reagent for modifying a nucleic acid; amplification primers; amplification enzyme; probes (e.g., labeled probes); etc.

In some embodiments, kits comprise the components of a membrane, such as the phospholipids needed to form an amphiphilic layer, such as a lipid bilayer. In some embodiments, kits comprise one or more other reagents or instruments to enable any of the embodiments mentioned above to be carried out. Such reagents or instruments include one or more of the following: suitable buffer(s) (aqueous solutions), components to obtain a sample from a subject (such as a vessel or an instrument comprising a needle), reagents to amplify and/or express polynucleotides, a membrane as defined above or a voltage or patch clamp apparatus. Reagents may be present in the kit in a dry state such that a fluid sample resuspends the reagents. The kit may also, optionally, comprise instructions to enable the kit to be used accordingly or details regarding which patients the technologies may be used for. The kit may, optionally, comprise nucleotides.

### Samples

The technology comprises analysis of any kind of nucleic acid sample. For example in some embodiments the sample comprises numerous types of nucleic acid molecules and it is apparent from the current measurements which are the molecules of interest. Alternatively, in some embodiments the nucleic acid molecules of interest are purified prior to passing them through the nanopore. For example, in some embodiments a sample originates from a biological source. Encompassed are biological fluids such as lymph, urine, cerebral fluid, bronchoalveolar lavage fluid (BAL), blood, saliva, serum, feces, or semen. Also encompassed are tissues, such as epithelium tissue, connective tissue, bones, muscle tissue such as visceral or smooth muscle and skeletal muscle, nervous tissue, bone marrow, cartilage, skin, mucosa or hair. In some embodiments, a sample is a sample originating from an environmental source, such as a plant sample, a water sample, an air sample, or a soil sample. In some embodiments, the sample originates from a household or industrial source; in some embodiments the sample originates from a food, beverage, cosmetic, or other composition or product that is intended for consumption by an animal (e.g., a human) and/or contact with an animal (e.g., a human). In some embodiments, a is a sample originating from a biochemical or chemical reaction or a sample originating from a pharmaceutical, chemical, or biochemical composition.

In some embodiments, the sample has a volume of 1000 μl or less, a volume of 500 μl or less, a volume of 100 μl or less, or a volume of 50 μl or less.

In some embodiments (e.g., in the case of solid samples or viscous suspensions), the sample is solubilized, homogenized, and/or extracted with a solvent prior to use in the present technology, e.g., to provide a liquid sample, e.g., having a lower viscosity and/or concentration of nucleic acid to be tested. In some embodiments, a liquid sample is a solution and in some embodiments a liquid sample is a suspension. In some embodiments, liquid samples are subjected to one or more pre-treatments prior to use in the present technology. Such pre-treatments include, but are not limited to dilution, filtration, centrifugation, pre-concentration, sedimentation, dialysis, lysis, elution, extraction, and precipitation. In some embodiments, pre-treatments include the addition of chemical or biochemical substances to the solution, such as acids, bases, buffers, salts, solvents, reactive dyes, detergents, emulsifiers, chelators, enzymes, and/or chaotropic agents.

The technology is related to characterizing a polynucleotide in a sample (e.g., a target, e.g., a target polynucleotide or a target nucleic acid). The technology comprises methods for characterizing a polynucleotide (e.g., a nucleic acid) without necessarily determining the nucleotide sequence of the polynucleotide, though embodiments provide characterization of a polynucleotide to supplement determination of or knowledge of a nucleotide sequence. A polynucleotide, such as a nucleic acid, is a macromolecule comprising two or more nucleotides. The polynucleotide or nucleic acid may comprise any combination of any nucleotides. The nucleotides can be naturally occurring or artificial. One or more nucleotides in the target polynucleotide can be oxidized or methylated. One or more nucleotides in the target polynucleotide may be damaged. For instance, the polynucleotide may comprise a pyrimidine dimer. One or more nucleotides in the target polynucleotide may be modified, for instance with a chemical moiety, label, or a tag. The target polynucleotide may comprise one or more spacers.

A nucleotide typically comprises a nucleobase, a sugar, and at least one phosphate group. The nucleobase is typically heterocyclic. Nucleobases include, but are not limited to, purines and pyrimidines and more specifically adenine, guanine, thymine, uracil, and cytosine. The sugar is typically a pentose sugar. Nucleotide sugars include, but are not limited to, ribose and deoxyribose. The nucleotide is typically a ribonucleotide or a deoxyribonucleotide. The nucleotide typically contains a monophosphate, a diphosphate, or a triphosphate. Phosphates may be attached on the 5′ or 3′ side of a nucleotide.

Nucleotides include, but are not limited to, adenosine monophosphate (AMP), guanosine monophosphate (GMP), thymidine monophosphate (TMP), uridine monophosphate (UMP), cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), and deoxycytidine monophosphate (dCMP). A nucleotide may be abasic (lack a nucleobase). A nucleotide may also lack a nucleobase and a sugar (a C3 spacer).

The nucleotides in the polynucleotide may be attached to each other in any manner. The nucleotides are typically attached by their sugar and phosphate groups as in nucleic acids. The nucleotides may be connected via their nucleobases as in pyrimidine dimers.

Embodiments provide that the polynucleotide is single stranded or double stranded. Particular embodiments provide that at least a portion of the polynucleotide is single stranded.

In some embodiments, the polynucleotide is a nucleic acid, such as deoxyribonucleic acid (DNA) or ribonucleic acid (RNA). The target polynucleotide can comprise one strand of RNA hybridized to one strand of DNA. The polynucleotide may be any synthetic nucleic acid known in the art, such as peptide nucleic acid (PNA), glycerol nucleic acid (GNA), threose nucleic acid (TNA), locked nucleic acid (LNA), or other synthetic polymers with nucleotide side chains.

The whole or only part of the target polynucleotide may be characterized using the technology. The target polynucleotide can be any length. For example, the polynucleotide can be at least 10, at least 50, at least 100, at least 150, at least 200, at least 250, at least 300, at least 400 or at least 500 nucleotide pairs in length. The polynucleotide can be 1000 or more nucleotide pairs, 5000 or more nucleotide pairs in length or 100000 or more nucleotide pairs in length.

The target polynucleotide is present in any suitable sample. The technology is typically carried out on a sample that is known to contain or suspected to contain the target polynucleotide. Alternatively, the technology may be carried out on a sample to confirm the identity of one or more target polynucleotides whose presence in the sample is known or expected.

### Uses

The technology finds use, e.g., without limitation, in nucleic acid forensics, repeat disease diagnostics, karyotyping, infectious disease identification, oncology, clinical chemistry diagnostics, and epigenomic studies among other examples. The technology finds use in applications that analyze nucleic acids. For instance, and without limitation, the technology finds use to characterize and/or identify forensic profiles. Many forensic applications benefit from fast processing associated with nanopore analysis of nucleic acids. In some particular applications, nucleic acid amplification (e.g., to amplify a target region comprising short tandem repeats (STRs)) is used to identify an individual based on the number of STR repeats in the target region. The forensic STR amplicons are typically small (roughly 20-500 nucleotides) and do not produce quality nucleotide sequence information by nanopore sequencing technologies. Embodiments of the present technology provide forensic analysis methods for identifying and counting forensic amplicons (e.g., STRs) on a nanopore device. In some embodiments, STR amplicons are concatenated to produce long nucleic acids that produce signals when translocated through a nanopore. In some embodiments, the concatamers comprise one or more modified nucleotides for detection of individual amplicons of the concatemer or comprise a distinguishable linker molecule that identifies the boundaries of each individual amplicon in the concatemer. In some embodiments, nanopore current and/or conductance data is analyzed to identify amplicons in a concatemer, e.g., using pattern recognition, peak fitting, or bioinformatics analysis to identify and/or separate and/or count amplicons after production of a current and/or conductance signal by nanopore translocation. For instance, embodiments provide use of modified nucleotides to recognize and count a repeat unit (e.g., an amplicon in a nucleic acid concatemer). As such, the modified nucleotides are counted without acquiring full nucleotide sequence information. In some embodiments, algorithms are provided to analyze patterns produced in real-time, e.g., to detect amplicons and provide base information, e.g., base count, base composition, base patterns, amplicon number, etc.

In some embodiments, the technology provides information about nucleic acid base composition, number, patterns, etc. (e.g., base counting and base composition). In some embodiments, the signals and information provided by the technology finds use in the identification of variable number tandem repeat (VNTR) sequences, STR sequences, polynucleotide regions, and other sequence types that produce errors in direct nanopore sequencing. For example, the technology finds use in characterizing forensic STRs. In some embodiments, the technology finds use in nucleic acid diagnostics, e.g., for Huntington disease, spinocerebellar ataxias, fragile X syndrome, myotonic dystrophy, juvenile myoclonic epilepsy, and Friedreich's ataxia. In some embodiments, modified nucleotides and nanopore analysis finds use to provide nucleic acid (e.g., DNA) tags for tracking and purity analysis of commercial products. In some embodiments, modified nucleotides and nanopore analysis finds use in RFLP analysis.

In additional embodiments, the technology finds use in identification of single nucleotide polymorphisms (SNPs), sequence rearrangements (e.g., inserts, deletions, translocations, breaks, fusions, inversions), etc. In some embodiments, the distinct signatures associated with modified nucleotides are analyzed with algorithms that are less complex than those associated with nucleotide sequencing (e.g., nanopore nucleotide sequencing). While the technology comprises use of any analysis, algorithm, or software code to analyze nanopore signals, in some embodiments the technology comprise use of less complex hardware and software relative to nucleotide sequencing, which reduces failure modes and costs (e.g., capital costs and time costs).

In some embodiments, the technology finds use in epigenomic and epigenetic analysis. For example, in some embodiments, the technology finds use in analysis of the methylation state of a nucleic acid. For example, in some embodiments, the technology finds use in DNA methylation analysis based on conversion of unmodified (e.g., unmethylated) cytosines to uracil using bisulfite reagent. See, e.g., Frommer (1992) “A genomic sequencing protocol that yields a positive display of 5-methylcytosine residues in individual DNA strands” Proceedings of the National Academy of Sciences of the United States of America. 89(5)1827-31. Methylated cytosines are not converted to uracil. Then, in some embodiments, an abasic site is produced in the nucleic acid by treating the nucleic acid comprising uracils with an enzyme that excises uracils from DNA, such as uracil-DNA glycosylase (UDG) or uracil N-glycosylase (UNG). Nanopore analysis is then used to analyze the number, location, pattern, etc., of uracils in the nucleic acid, which provides information about the number, location, pattern, etc. of methylated and unmethylated cytosines in the original nucleic acid.

In some embodiments, the technology finds use in rapid identification of viral or microbial pathogens (e.g., monitoring ebola), environmental monitoring, food safety monitoring, monitoring of antibiotic resistance, and haplotyping.

In some embodiments, the technology relates to creating unique and/or detectably distinct signals when nucleic acids comprising the described modifications translocate through a nanopore. While the modifications are used in various embodiments individually or combinatorially to analyze nucleic acids where full nucleotide sequence determination is not required, the modifications are used in some embodiments individually or combinatorially to analyze nucleic acids in addition to determination of nucleotide sequence.

Embodiments provide that any base modification finds use in this technology, e.g., to resolve any homopolymer stretch of interest using nanopore sequencing technologies. In some embodiments, the technology finds use in, e.g., forensic and diagnostic testing along with any situation that would benefit from an improvement in resolving homopolymer stretches. Additional uses of the technology include, but are not limited to, karyotyping, sequencing short amplicons, regulating of sequencing speed and/or activity. The technology also finds use in, e.g., determining nucleotide sequences without base calling, detecting DNA hydroxymethylation, and for use as a calibration standard provide calibration between samples, pores, devices, or days.

### EXAMPLES

### Example 1

### Detecting Repeat Sequences in a Nucleotide Sequence

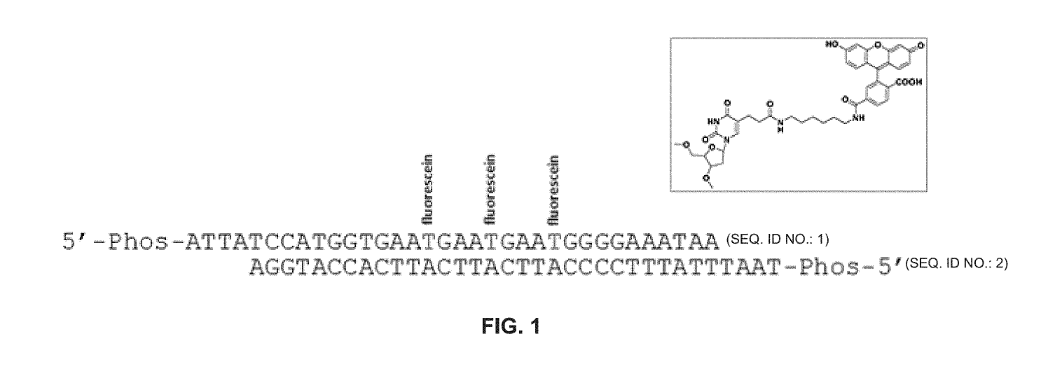

During the development of embodiments of the technology described herein, experiments were conducted in which a nucleic acid was designed and synthesized to comprise three tetranuclotide repeats of GAAT sequence flanked by neighboring regions to create an oligonucleotide having a total length of 34 nucleotides. The thymidine of each GAAT repeat was modified with a linker and a covalently-attached fluorescein dye moiety. A second nucleic acid comprising a sequence complementary to a portion of the first nucleic acid was also designed and synthesized. The second nucleic acid was unmodified (e.g., the second nucleic acid comprised no modified nucleotides) and also had a total length of 34 nucleotides and. The second nucleic acid was designed to anneal with the first nucleic acid to form a duplex between the complementary regions and a four-nucleotide 5′ single-stranded region (e.g., overhang) on each end (see, e.g., FIG. 1).

The 5′ nucleotide overhangs with phosphates were added so that the double stranded product could be ligated to other nucleic acids to provide a longer nucleic acid and thus to provide longer read lengths during nucleic acid translocation through the nanopore. While longer nucleic acids generally provide higher quality data in nanopore processing and data analysis, data collected using the double stranded product alone, e.g., without ligating it to another nucleic acid (e.g., shown in FIG. 1), were of high quality, thus indicating that a ligation strategy is not required to produce high quality data. However, embodiments of the technology are contemplated in which ligation of a nucleic acid to another nucleic acid is used. Such a strategy finds use, for example, in embodiments of the technology comprising use of nucleic acid amplification that produces short and different amplicons; and in embodiments of the technology comprising use of nucleic acids as reporter or barcoding oligonucleotides that are attached to primers and later released via chemical or enzymatic methods.

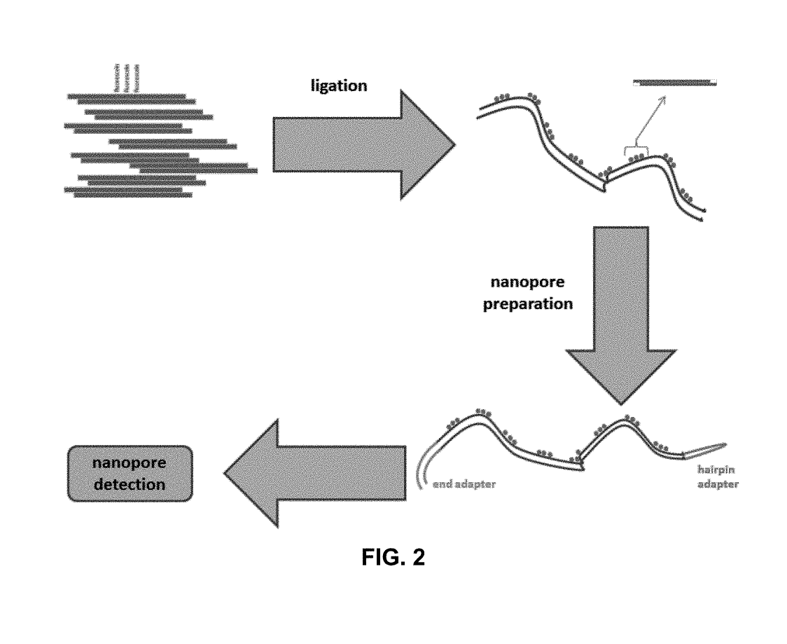

To test the signal produced by the modified nucleic acid in a nanopore sequencing apparatus, the short duplexes were ligated together to produce longer nucleic acids appropriate for processing by a commercial library preparation kit. Preparation of the library included adding an adapter to the 5′ end of the ligated construct and a hairpin adapter to the other end of the ligated construct, which provided a nucleic acid that was compatible with the commercial nanopore sequencer used for the experiments. A flow diagram of the process is illustrated in FIG. 2.

Following sample preparation (e.g., as shown in FIG. 2), the sample was loaded onto the nanopore device and the current of the ionic solution was continuously monitored.

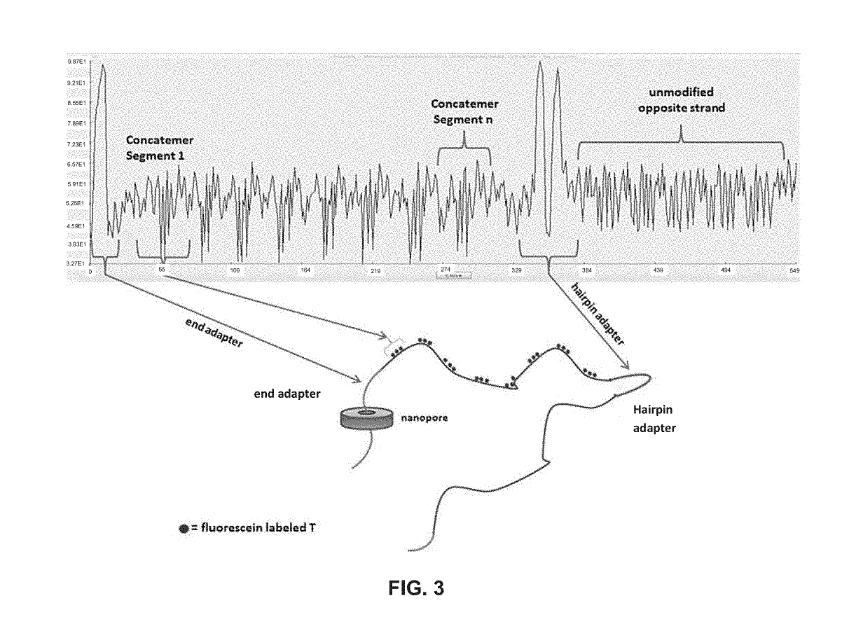

A portion of the raw data collected is shown in FIG. 3. The concatemer sequence has a 5′ adapter that initiates translocation of the nucleic acid through the pore and a hairpin adapter that provides for translocation of the complementary strand through the nanopore. Both the 5′ adapter and the hairpin adapter display a unique high current signal in the raw data (FIG. 3). When the fluorescein-modified thymidines passed through the nanopore, a significant reduction in current was observed for each modification (FIG. 3). Furthermore, a triplet signal was observed for each triply-labeled tetranucleotide repeat (FIG. 3). Thus, the data collected during the testing of embodiments of the technology indicated that a clear and unambiguous signal identified the number of thymidines in each repeat. From these data, the number of repeats in each concatemer segment was determined without knowledge of the nucleotide sequence of the nucleic acid tested. The data collected indicated that the type (e.g., quality, e.g., increase or decrease) of change and the magnitude (e.g., quantity) of change in the nanopore ion current was related to the modification of the nucleotide. Additionally, the translocation time (or change in translocation time) of the modification through the nanopore also provides a significant and distinct signature in the data. Accordingly, the data indicate that the type of modified nucleotide provides a technology for the analysis of complex repeats or base counting of alternative sequences. Furthermore, this type of signal reduces and/or minimizes the need for nucleotide sequence information in some applications and thus provides a technology for repeat counting, single nucleotide polymorphism determination, gene alignment determination, etc., and other analyses that do not require sequencing.

The data indicated that the signal produced by the modified nucleotide provided a basis for counting nucleotide repeats. However, the technology contemplates other modifications that provide distinct current signals for each type of modified nucleotide (or set of nucleotides), which thus provides technologies for addressing complex nucleic acid analysis. In exemplary embodiments, incorporating modifications occurs during a primer extension process, by using modifications directly compatible with enzymatic amplification reactions, or using other post-amplification or synthesis processing. Additionally, modifications to primers associated with amplification strategies, resulting in distinct nanopore current signals, find use in identifying individual products and starting points in concatenation sequences. In some embodiments, the number and type of modifications is scaled to detect multiple products or to barcode multiple samples within a single nanopore run.

### Example 2

### Abasic Nucleotide Residues

During the development of embodiments of the technology, experiments were conducted to test the signal detected for a nucleic acid comprising an abasic site in a nanopore device. Abasic sites are sites in a nucleic acid that contain no base (e.g., the site is apyrimidinic or apurinic). Abasic sites are sterically hindered much less than normal nucleotides and thus are contemplated to behave differently than normal nucleotides when translocating through a nanopore.

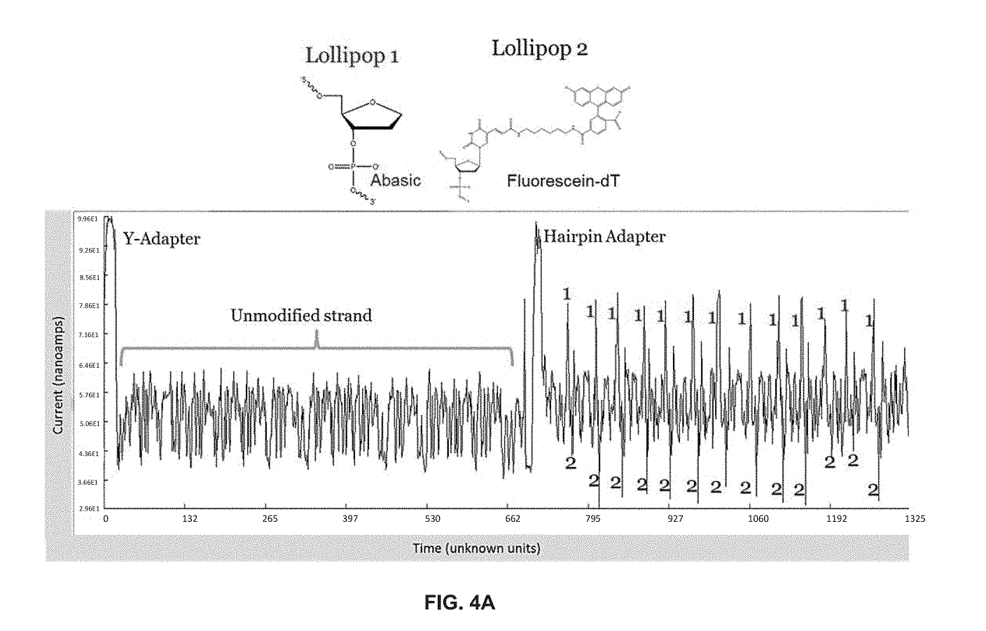

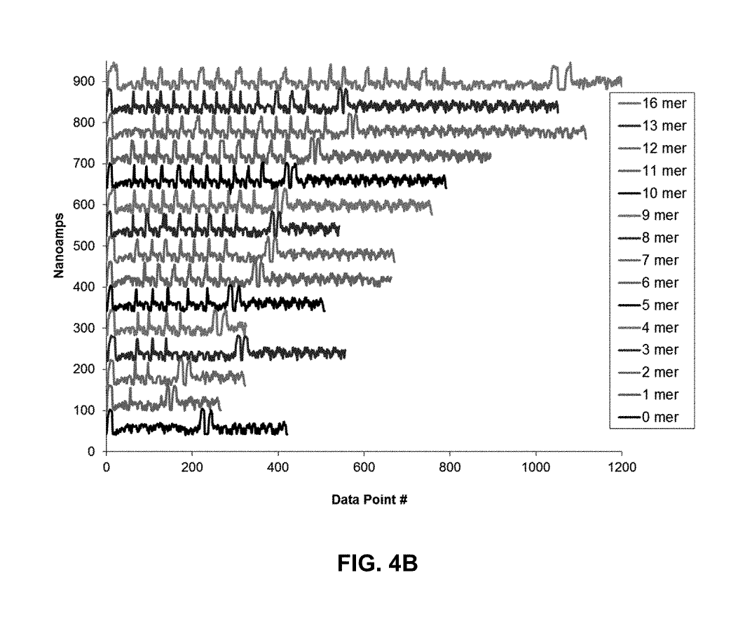

Abasic sites are readily detectable in a nucleic acid using a nanopore device (FIG. 4A and 4B). In particular, data were collected indicating that abasic sites produce a large positive peaks in current (FIG. 4A).

Because, in some embodiments, analysis of a nucleic acid using a nanopore provides temporal information, the technology provides for manipulating the number and order of abasic sites (e.g., combinatorially) to produce distinct signatures for identifying nucleic acids within a single sample, e.g., as a multiplex technique for identifying multiple nucleic acids based on their signatures.

For example, data were collected from nucleotides comprising multiple nucleotide modifications (e.g., abasic sites) on a single molecule (FIG. 4B). Nucleic acids comprising 0 to 16 abasic sites (a “site” consisted of 3 consecutive abasic nucleotide residues) were analyzed using a nanopore device. The data indicated an increase in current as the abasic site passed through the nanopore; multiple sets of abasic sites were easily be distinguished on the same molecule (FIG. 4B). Accordingly, the data indicated that the abasic site approach finds use in determining base count and composition.

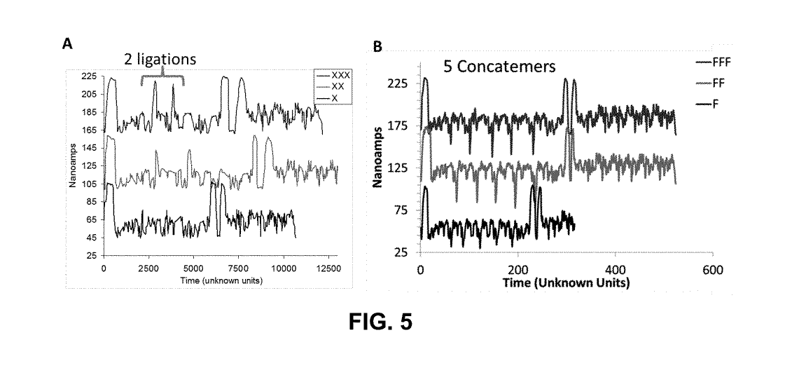

Furthermore, during the development of embodiments of the technology, experiments were conducted to test the signal produced by a nucleic acid comprising modifications at multiple consecutive sites. Nucleic acids were produced to comprise abasic sites or fluorescein moieties at 1, 2, or 3 consecutive sites. The data show that increasing the number of consecutive modified sites increases the magnitude and width of the signal (FIG. 5).

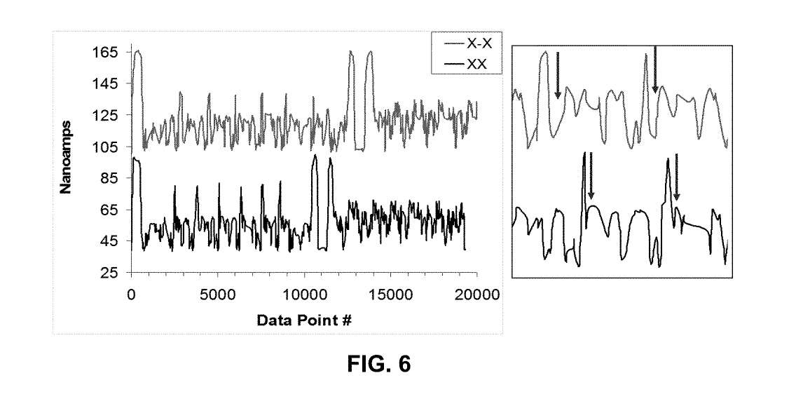

In additional experiments, data collected indicated that the signal varied as a function of the spacing of modifications in a nucleic acid. In particular, two consecutive abasic sites produced a different nanopore current signature than two abasic sites separated by one unmodified base between them (FIG. 6).

These data indicated that the number of consecutive modified nucleotides and/or the spacing between modified nucleotides can be varied to modulate the signal quality (peak spacing, peak shape) and signal quantity (e.g., magnitude, e.g., increase or decrease in signal). The modifications produced recognizable patterns in the nanopore current signal, which were used to detect and/or count individual bases. In an exemplary contemplated use of the technology, modified bases find use in providing barcodes to identify samples.

### Example 3

### Multiplex Modification and Detection

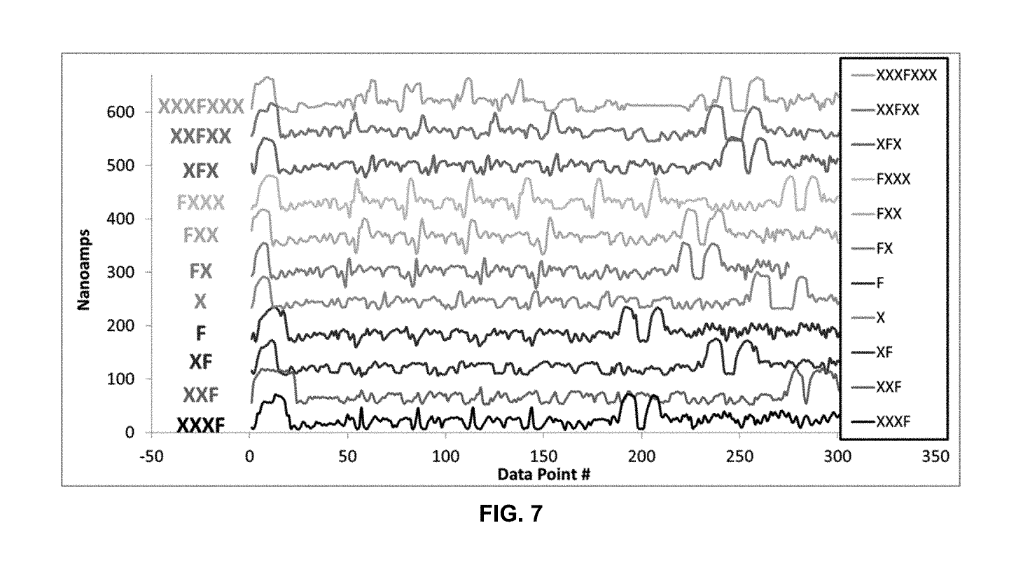

During the development of embodiments of the technology, data were collected indicating that combining nucleotide modifications on the same nucleic acid molecule produce distinct nanopore current signatures. For example, experiments were conducted in which combining fluorescein-modified nucleotides and abasic sites in combinatorial patterns produced multiple distinct current signatures (FIG. 7). Each combination produced a distinct signal that identified the molecule. Accordingly, the technology comprises multiplex applications in which multiple tests are performed in a single reaction or for barcoding individual samples that are pooled prior to analysis on the nanopore system.

### Example 4

### Nucleotide Modifications

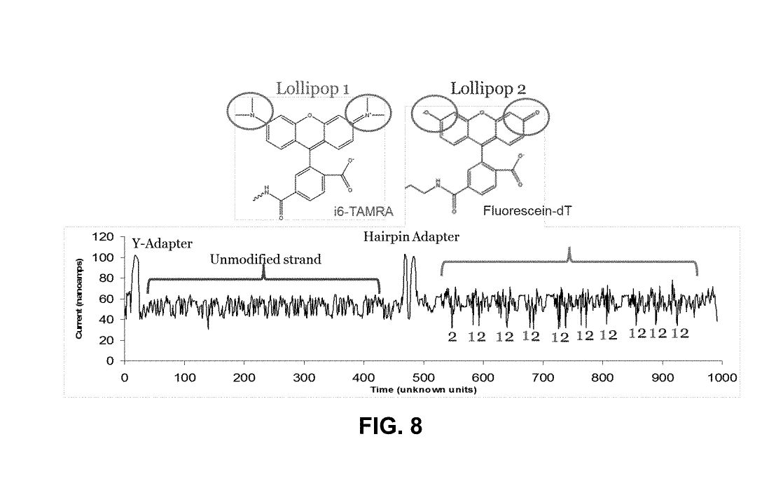

During the development of embodiments of the technology described herein, multiple types of nucleotide modifications were tested in addition to modification with fluorescein and introduction of abasic sites described above. In particular, data were collected from experiments in which nucleotides were modified with other moieties, e.g., other fluorescent dyes such as, e.g., TAMRA dye (carboxytetramethylrhodamine) TAMRA has a structure similar to fluorescein and produces a signal similar to fluorescein. Experiments were conducted in which a nucleic acid was produced to comprise nucleotides modified with fluorescein and TAMRA dye (FIG. 8). The nanopore signals produced by TAMRA dye and fluorescein were similar (FIG. 8).

Accordingly, the technology comprises use of any modification that produces a signal (or a change in a signal) when a nucleic acid comprising the modification is translocated through a nanopore. In some embodiments, the modification is a modification of a nucleotide (e.g., covalent attachment of a moiety to a nucleotide, creation of an abasic nucleotide residue, etc.). In some embodiments, the modification is modification of the structure of the nucleic acid itself (e.g., peptide nucleic acid bonds between nucleotides, linker moieties between nucleotides, phosphorothioate bonds between nucleotides, etc.). For example, the technology comprises use of the following nucleic acid and nucleotide modifications to produce detectable signals in a nanopore device.

In some embodiments, nucleic acids comprise a DNA spacer. DNA spacers are chemical modifications of a nucleic acid that create distance between two nucleic acid segments in a sequence. For example, DNA spacers include but are not limited to commercially available spacers based on phosphoramidite, hexanediol, triethylene glycol, and hexa-ethyleneglycol. The addition of a DNA spacer to a nucleic acid creates a gap in the nucleotide sequence that changes the interaction of the nucleic acid with the nanopore and changes (e.g., increases) the conductance of the nanopore, which produces a change in the signal data. Spacers are available having different lengths (e.g., in some embodiments, spacers produce a distance between consecutive nucleotides that is smaller than the natural linkage between nucleotides; in some embodiments, spacers produce a distance between consecutive nucleotides that is equal to or greater than the natural linkage between nucleotides). During translocation of the nucleic acid through the nanopore, the signal provides temporal information (e.g., time domain) in addition to the quality and magnitude of the electrical (e.g., conductance, current, resistance, voltage, impedance, etc.) signal. Thus, spacers of different lengths produce distinct temporal signatures in the data, e.g., for combinatorial analysis.

During the development of embodiments of the technology, data were collected from experiments testing nucleic acids comprising spacers of varying types and lengths. In particular, experiments were conducted using a C9 spacer (three consecutive C3 spacers) and a polyethylene glycol (PEG) spacer. The C9 produced a large positive peak of current (FIG. 9). The PEG18 spacer also produced a large positive peak, but translocation of the nucleic acid comprising the PEG18 spacer was hindered (FIG. 10). The technology contemplates other spacers that have similar signals.

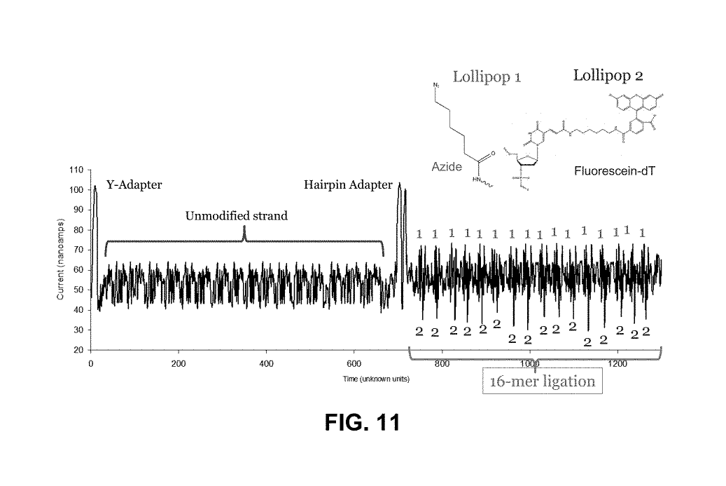

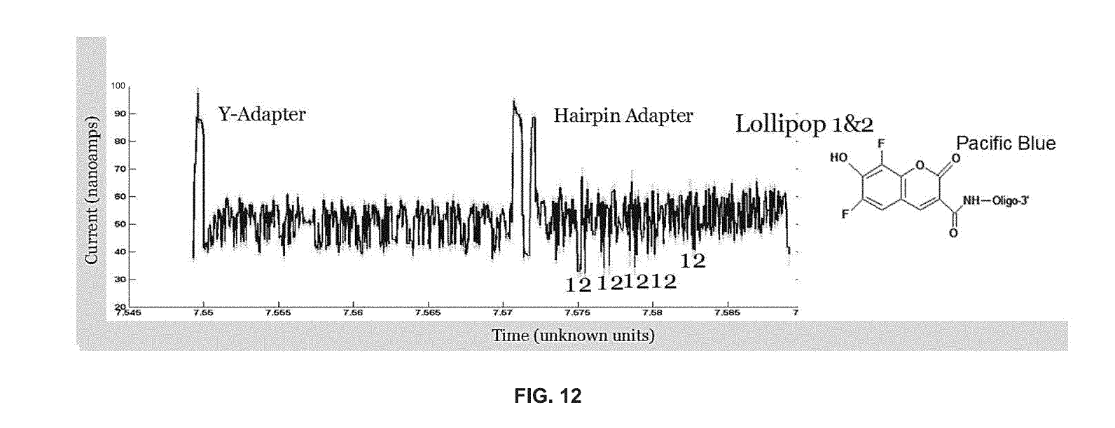

In some embodiments, nucleic acids comprise a nucleotide modified chemically (e.g., in some embodiments nucleic acids comprise a chemically-modified nucleotide, e.g., in some embodiments nucleic acids comprise a nucleotide comprising a chemically-modified base, sugar, or phosphate). For instance, chemicals such as biotin, azide, glucose, and EDTA are added to a nucleotide base to create a modified nucleotide detectable by a nanopore. During the development of embodiments of the technology, data were collected that indicated that chemical modification of a nucleotide base produced a distinct signal for a nucleic acid comprising the modification when it translocated through a nanopore. In particular, data were collected for a nucleic acid modified by azide (FIG. 11) for a nucleic acid modified with PACIFIC BLUE dye (FIG. 12).

### Example 5

### Detection of Immunoreactions

In some embodiments, the technology relates to immunoreaction assays (e.g. comprising recognition of an antigen by an antibody or antigen-binding fragment thereof), such as those used in diagnostic testing. Currently, immunoreactions are detected using enzymatic or PCR-based methods. However, these detection methods are limited by their dynamic range and specificity. Some embodiments of the technology described herein utilize the high specificity of antibody-antigen interactions combined with the precision of nucleic acid analysis on a nanopore sequencer. These embodiments of the technology provide a robust and efficient detection of immunoreactions on a nanopore sequencer. For example, in some embodiments the technology comprises modified oligonucleotides in a barcode-like schema that are detected in a nanopore system without the need for determining the full DNA sequence of the barcode. In some embodiments, a double-stranded (e.g., duplex) oligonucleotide is conjugated to an antibody and or antibodies and then one strand melted off with temperature or chemicals to allow for detection by nanopore analysis. Additionally, in some embodiments the DNA attached to antibodies is first enzymatically amplified (e.g. PCR, isothermal methods, etc.) and then modified with nucleotide modifications as described herein for analysis via nanopore.

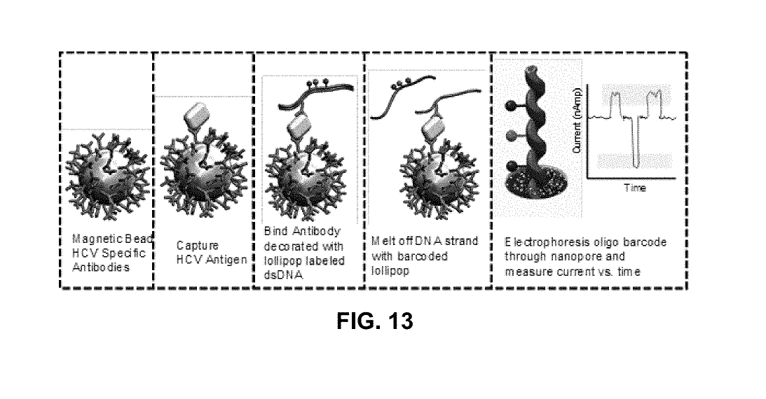

In particular embodiments, the technology combines nanopore analysis of nucleic acids with immunoreactions, which are highly specific reactions that allow detection of antigens and epitopes (e.g., pathogens such as hepatitis or HIV). In some embodiments, the technology comprises use of magnetic beads coated with antigen-specific antibodies to capture the antigen. Once captured and washed, a second antigen-specific antibody conjugated to a strand of double-stranded nucleic acid (e.g., DNA) is introduced. This second antibody comprises a nucleic acid comprising a strand that is a modified nucleic acid and/or that is a nucleic acid comprising modified nucleotides that is appropriate for analysis (e.g., detection, identification, characterization) in a nanopore device by the associated signature in the current and/or conductance versus time. After washing away unbound antibody, the double-stranded oligonucleotide can be melted using methods such as heat, alkalinity (e.g., NaOH), enzymatic cleavage, and/or other chemical or biochemical methods to release the single strand comprising modifications. The modified strand is then prepared for nanopore analysis or, in some embodiments, the modified strand already comprises the adapters for analysis through a nanopore. For instance, in some embodiments the melted strand mimics or comprises the self-complimentary hairpin adapter. The nucleic acid and/or nucleotide modifications provide for analysis of base composition and/or number (e.g., base counting) rather than nucleotide sequence determination, thus providing a technology to measure levels of pathogen quickly and easily without the need for sequence determination by sequencing. See, e.g., FIG. 13.

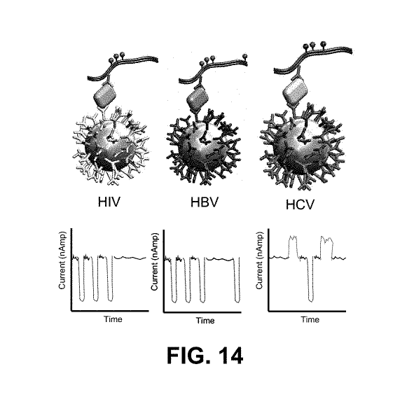

In some embodiments, the technology provides “barcodes”, e.g., produced by combinatorial modifications of nucleic acid-antibody probes. During the development of embodiments of the technology provided herein, an oligonucleotide was modified to comprise both a C9 spacer (e.g., a triplicate C3 spacer) and a fluorescein moiety. As described above, the C9 spacer reduces impedance in the nanopore and thus increases current flow. As described above, the fluorescein molecule increases impedance in the nanopore and thus decreases current flow. Data collected from nanopore analysis of the C9/fluorescein-modified nucleic acid in the nanopore indicated that both modifications were detected (FIG. 9). See, e.g., Example 4. In an exemplary embodiment for pathogen detection, the number of modifications are counted (in real-time as the molecule passes through a nanopore) to provide a fast and accurate count of pathogen levels. In some embodiments, the reactions are multiplexed by using modifications that produce distinct signatures. For example, FIG. 14 shows a schematic where HIV, HBV, and HCV each have a distinct dsDNA-antibody probe comprising an individually recognized modification. The individual pathogen reporter sequences for each probe are counted to obtain an absolute number or to obtain a relative count number based on internal standards. Multiplexing provides a screen for a large number of different pathogens in a single test. This technology, in some embodiments, thus improves (e.g., increases the accuracy, speed, and/or portability) of immune-based diagnostic reactions.

### Example 6

### Detection of Small Nucleic Acids

In some embodiments, the technology relates to distinguishing and counting small nucleic acids (e.g., small RNAs such as, e.g., miRNA, piRNA, tRNA, ncRNA, etc.) in a sample. For example, small RNA is increasingly recognized as an important biomarker of cellular health, and rapid detection of small RNA within a sample provides a useful diagnostic assay based on these molecules. Extant nanopore methods, such as nanopore sequencing, are optimized for long strands of nucleic acid and thus are not appropriate for analysis of a small RNA. As described herein, nucleic acid modifications, e.g., modified nucleotides, provide a significant perturbation to nanopore conductance relative to unmodified nucleotides. In some embodiments, the change in the electrical signal, in the time domain and/or in magnitude, is controlled by the size, structure, and/or charge of the nucleic acid modification.

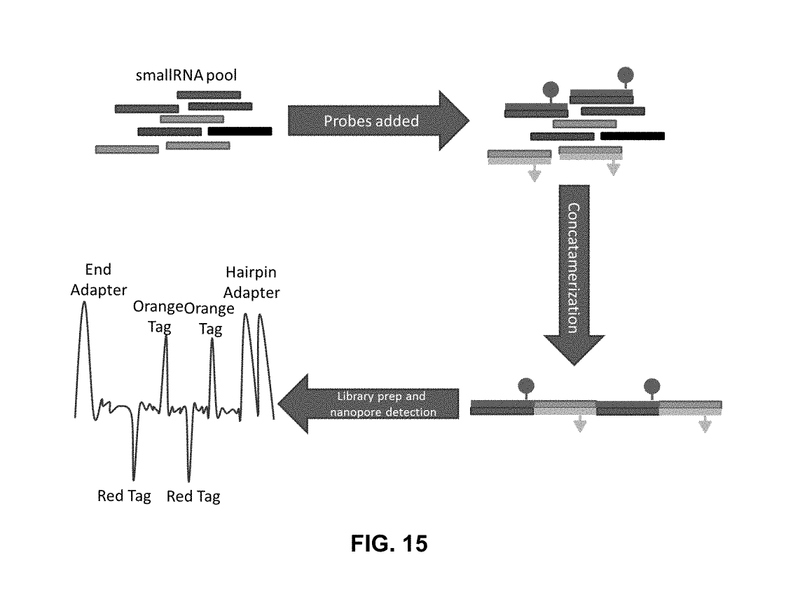

This technology finds use in analysis of small nucleic acids, e.g., by using a modified anti-small RNA probe that binds (hybridizes) to its corresponding small RNA target. In some embodiments, the technology comprises an optional step of concatamerizing hybridized probe-target reporter molecules for counting as the concatemer passes through a nanopore. In some embodiments, the technology comprises undergo library preparation and analysis on a commercial nanopore device. The probe molecule provides a distinct signal for an individual small RNA molecule by comprising a distinct nucleotide modification. In some embodiments, the probe comprises an uncharged oligonucleotide backbone (e.g., PNA) so that the probe does not translocate through the nanopore alone but will translocate through the nanopore when hybridized to a small RNA. PNA molecules only ligate with other molecules as double stranded nucleic acid and pass through a nanopore when concatamerized with charged molecules. Embodiments also provide probes with charged backbones. In some embodiments, concatamerization is controlled so that only double stranded molecules are ligated, thereby greatly enriching for targets hybridized to probes. Therefore, signals from nucleic acids translocating through the nanopore are produced by probe-target hybridized complexes. Multiplex embodiments provide multiple distinguishable modified probes for use in parallel to identify multiple target small RNAs and control (normalization) small RNAs (see, e.g., FIG. 15).

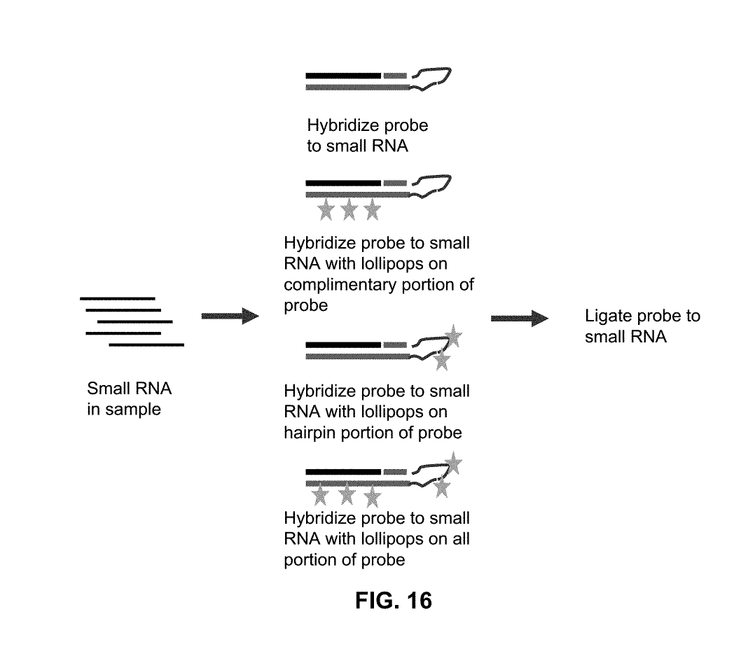

In some embodiments, the technology comprises ligating a small RNA to a modified or unmodified complimentary probe. In some embodiments, the probe comprises a portion of sequence that is not complementary to the sequence of the small RNA target that is ligated to the RNA strand (FIG. 16). In some embodiments, the probe has a hairpin structure that mimic a commercial hairpin adapter and includes modifications to identify the probe sequence as either a hairpin or for barcoding. In some embodiments, probes comprise multiple modifications (e.g., chemical modifications, abasic sites, spacers). In some embodiments, the probes comprise other modifications such as biotin for aid in purification.

Using this technology, small RNA species are quantified by counting particular modifications associated with particular small RNA targets and bound probes. In addition, in some embodiments counts are compared to counts for a control small RNA that does not change the nanopore signal to provide quantification across experiments.

### Example 7

### Use of Mixed Bases to Resolve Homopolymeric Regions

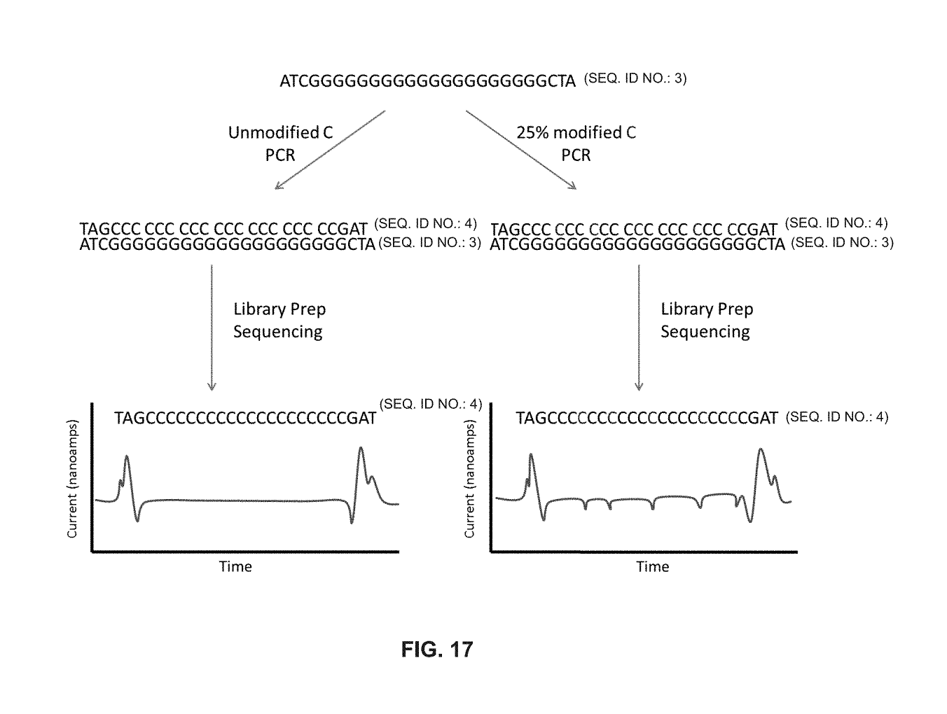

Due to the inherently reduced complexity of homopolymer stretches, current nanopore sequencing technologies have difficulty in distinguishing the sequence and/or length of the stretch. Accordingly, during the development of embodiments of the technology described herein, experiments were conducted in which modified bases were used that produce unique signatures for a homopolymeric stretch passing through a nanopore. In these experiments, base calling algorithms were used to resolve short stretches of homopolymers while still maintaining the integrity of the sequence. In particular, data were collected from experiments in which modified bases were introduced into a nucleic acid during PCR amplification. The resulting amplification product comprised one or more modified bases (including but not limited to methylation) at random locations throughout homopolymer stretches, e.g., a fraction of the bases of the amplification product comprised a modification. In some embodiments, the relative fraction of modified bases to unmodified bases (e.g., 0.1, 0.15, 0.2, 0.25, 0.3, 0.35, 0.4, 0.45, 0.5, 0.6, 0.65, 0.7, 0.75, 0.8, 0.85, 0.9, 0.95) is varied to find a fraction that maximizes the resolution of a particular homopolymeric region of interest (see, e.g., FIG. 17).

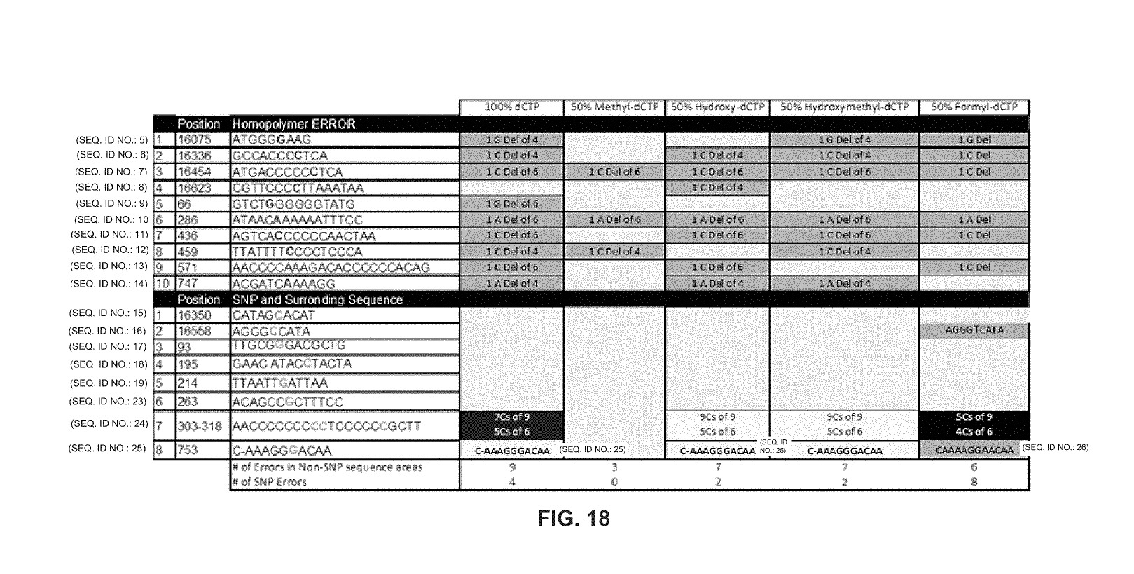

Data collected from experiments conducted during the development of embodiments of the technology using modified bases in homopolymeric regions indicated that sequencing errors in homopolymer stretches were reduced. In particular, a ratio of modified to un-modified cytosine bases of approximately 0.50 in a poly-C region (e.g., 50% methyl- dCTP) reduced sequencing errors in the homopolymeric region (FIG. 18). Some embodiments of the technology were tested in which bases were modified with hydroxy, hydroxymethyl, or formyl groups alone or in combination. The data indicated that these other mixtures of modified bases (e.g., hydroxyl, hydroxymethyl, formyl, etc.) also provide improved sequencing results when used with standard bases in nanopore sequencing. While the data collected were for experiments testing modification of dCTP, the technology encompasses modification also of A, G, T, and U bases, in addition to non-standard or other non-traditional DNA or RNA bases. In some embodiments, mixed natural and modified bases are used in combination with each other as well (e.g., a mixture of 50% methyl-dCTP, methyl-dATP, methyl-dGTP, and methyl-dTTP / methyl-dUTP mixed with 50% natural dCTP, dATP, dGTP, and dTTP / dUTP.

Embodiments provide that any base modification finds use in this technology, e.g., to resolve any homopolymer stretch of interest using nanopore sequencing technologies. In some embodiments, the technology finds use in, e.g., forensic and diagnostic testing along with any situation that would benefit from an improvement in resolving homopolymer stretches. Additional uses of the technology include, but are not limited to, karyotyping, sequencing short amplicons, regulating of sequencing speed and/or activity. The technology also finds use in, e.g., determining nucleotide sequences without base calling, detecting DNA hydroxymethylation, and for use as a calibration standard provide calibration between samples, pores, devices, or days.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1-68. (canceled)

69. A method for characterizing a nucleic acid, the method comprising:
a) modifying the nucleic acid to provide a modified nucleic acid;
b) translocating the modified nucleic acid through a nanopore;
c) measuring an electrical signal produced by translocation of the modified nucleic acid through the nanopore; and
d) characterizing the nucleic acid by analyzing the electrical signal.

70. The method of claim 69 wherein characterizing the nucleic acid comprises determining an unordered base composition of the nucleic acid.

71. The method of claim 69 wherein the electrical signal is current.

72. The method of claim 69 further comprising providing a voltage across the nanopore.

73. The method of claim 69 wherein modifying the nucleic acid comprises modifying a nucleotide of the nucleic acid; incorporating a modified nucleotide into the nucleic acid; modifying a linkage between nucleotides of the nucleic acid; linking a chemical moiety to a nucleotide; linking a dye to a nucleotide; providing an uncharged linkage between nucleotides of the nucleic acid; providing a linker between nucleotides of the nucleic acid; providing peptide nucleic acid linkage between nucleotides of the nucleic acid; and/or removing a base to produce an abasic site.

74. The method of claim 69 wherein characterizing the nucleic acid comprises identifying the presence of repeats in the nucleic acid.

75. The method of claim 69 wherein characterizing the nucleic acid comprises identifying the presence of a single nucleotide polymorphism in the nucleic acid.

76. The method of claim 69 wherein characterizing the nucleic acid comprises identifying the presence of a modified base in the nucleic acid.

77. A reaction mixture comprising a modified nucleic acid and a nanopore.

78. The reaction mixture of claim 77 wherein the modified nucleic acid comprises a modified nucleotide.

79. The reaction mixture of claim 77 wherein the modified nucleic acid comprises a modified linkage between two nucleotides, a chemical linker between two nucleotides, an abasic site, or a nucleotide modified with a covalently attached dye or other chemical moiety.

80. The reaction mixture of claim 77 wherein the nanopore comprises a protein.

81. The reaction mixture of claim 77 wherein the nanopore is a solid state nanopore.

82. The reaction mixture of claim 77 wherein a lipid bilayer comprises the nanopore.

83. A system comprising;
i) a nanopore apparatus; and
ii) a composition comprising:
a) a reagent to modify a nucleic acid; and/or
b) a modified nucleotide.

84. The system of claim 83 further comprising an electrical source providing a voltage or current.

85. The system of claim 83 further comprising a processor configured to analyze electrical signals recorded as a function of time.

86. The system of claim 83 wherein the reagent produces an abasic site in a nucleic acid; the composition comprises uracil-DNA glycosylase or uracil N-glycosylase; the composition comprises a reactive dye; the composition comprises a chemical linker or spacer; the reagent produces a chemically-modified nucleotide; the composition comprises an exonuclease; and/or the composition comprises a drag tag.

87. The system of claim 83 wherein the nanopore is a solid state nanopore or a protein nanopore.

88. The system of claim 83 further comprising a processor configured to perform a method for characterizing a nucleic acid.
